(19) United States
(12) Patent Application Publication
(10) Pub. No.: US 2018/0215781 A1
(43) Pub. Date: Aug. 2, 2018

(54) BETUIN DERIVATIVES FOR PREVENTING OR TREATING HIV INFECTIONS

(71) Applicant: GLAXOSMITHKLINE INTELLECTUAL PROPERTY (NO.2) LIMITED, Brentford, Middlesex (GB)

(72) Inventor: Brian Alvin JOHNS, Research Triangle Park, NC (US)

(21) Appl. No.: 15/745,521

(22) PCT Filed: Jul. 26, 2016
(86) PCT No.: PCT/IB2016/054458

§ 371 Date: Jan. 17, 2018

Related U.S. Application Data
(60) Provisional application No. 62/197,765, filed on Jul. 28, 2015.

(51) Int. Cl. C07J 63/00; A61P 31/18; A61K 45/06

(52) U.S. Cl. CPC C07J 63/008; A61K 45/06; A61P 31/18

(57) ABSTRACT

The present invention relates to compounds characterized by having a structure according to the following Formula I:

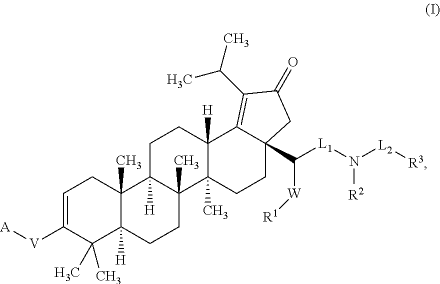

or a pharmaceutically acceptable salt thereof. Compounds of the present invention are useful for the treatment or prevention of HIV.

### FIELD OF THE INVENTION

The present invention relates to compounds, pharmaceutical compositions, and methods of use thereof for (i) inhibiting HIV replication in a subject infected with HIV, or (ii) treating a subject infected with HIV, by administering such compounds.

### BACKGROUND OF THE INVENTION

Human immunodeficiency virus type 1 (HIV-1) leads to the contraction of acquired immune deficiency disease (AIDS). The number of cases of HIV continues to rise, and currently over twenty-five million individuals worldwide suffer from the virus. Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still required because of undesirable drug-drug interactions; drug-food interactions; non-adherence to therapy; and drug resistance due to mutation of the enzyme target.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur. The emergence of multidrug-resistant HIV-1 isolates has serious clinical consequences and must be suppressed with a new drug regimen, known as salvage therapy.

Current guidelines recommend that salvage therapy includes at least two, and preferably three, fully active drugs. Typically, first-line therapies combine three to four drugs targeting the viral enzymes reverse transcriptase and protease. One option for salvage therapy is to administer different combinations of drugs from the same mechanistic class that remain active against the resistant isolates. However, the options for this approach are often limited, as resistant mutations frequently confer broad cross-resistance to different drugs in the same class. Alternative therapeutic strategies have recently become available with the development of fusion, entry, and integrase inhibitors. However, resistance to all three new drug classes has already been reported both in the lab and in patients. Sustained successful treatment of HIV-1-infected patients with antiretroviral drugs will therefore require the continued development of new and improved drugs with new targets and mechanisms of action.

Presently, long-term suppression of viral replication with antiretroviral drugs is the only option for treating HIV-1 infection. To date, a number of approved drugs have been shown to greatly increase patient survival. However, therapeutic regimens known as highly active antiretroviral therapy (HAART) are often complex because a combination of different drugs must be administered to the patient to avoid the rapid emergence of drug-resistant HIV-1 variants. Despite the positive impact of HAART on patient survival, drug resistance can still occur.

The HIV Gag polyprotein precursor (Pr55Gag), which is composed of four protein domains—matrix (MA), capsid (CA), nucleocapsid (NC) and p6—and two spacer peptides, SP1 and SP2, represents a new therapeutic target. Although the cleavage of the Gag polyprotein plays a central role in the progression of infectious virus particle production, to date, no antiretroviral drug has been approved for this mechanism.

In most cell types, assembly occurs at the plasma membrane, and the MA domain of Gag mediates membrane binding. Assembly is completed by budding of the immature particle from the cell. Concomitant with particle release, the virally encoded PR cleaves Gag into the four mature protein domains, MA, CA, NC and p6, and the two spacer peptides, SP1 and SP2. Gag-Pol is also cleaved by PR, liberating the viral enzymes PR, RT and IN. Gag proteolytic processing induces a morphological rearrangement within the particle, known as maturation. Maturation converts the immature, donut-shaped particle to the mature virion, which contains a condensed conical core composed of a CA shell surrounding the viral RNA genome in a complex with NC and the viral enzymes RT and IN. Maturation prepares the virus for infection of a new cell and is absolutely essential for particle infectivity.

Bevirimat (PA-457) is a maturation inhibitor that inhibits the final step in the processing of Gag, the conversion of capsid-SP1 (p25) to capsid, which is required for the formation of infectious viral particles. Bevirimat has activity against ART-resistant and wild-type HIV, and has shown synergy with antiretrovirals from all classes. Bevirimat reduced HIV viral load by a mean of 1.3 log₁₀/mL in patients who achieved trough levels of >=20 μg/mL and who did not have any of the key baseline Gag polymorphisms at Q369, V370 or T371. However, Bevirimat users with Gag polymorphisms at Q369, V370 or T371 demonstrated significantly lower load reductions than patients without Gag polymorphisms at these sites.

Other examples of maturation inhibitors can be found in PCT Patent Application No. WO2011/100308, PCT Patent Application No. PCT/US2012/024288, Chinese PCT Application No. PCT/CN2011/001302, Chinese PCT Application No. PCT/CN2011/001303, Chinese PCT Application No. PCT/CN2011/002105, PCT/CN2011/002159, WO2013/090664, WO2013/123019, WO 2013/043778, WO 2014/123889, WO 2011/153315, WO 2011/153319, WO 2012/106188, WO 2012/106190, WO 2013/169578, WO 2014/13081. Some previous maturation inhibitors have left open gaps in the areas of polymorphism coverage whereby potency against a broad range of clinically relevant gag sequences is important, along with overall potency including the clinically relevant protein adjusted antiviral activity that is helpful for robust efficacy in long term durability trials.

It would therefore be an advance in the art to discover alternative compounds that are an effective balance of the aforementioned properties for the prevention and/or treatment of HIV infections.

### SUMMARY OF THE INVENTION

In accordance with one embodiment of the present invention, there is provided a compound of Formula I:

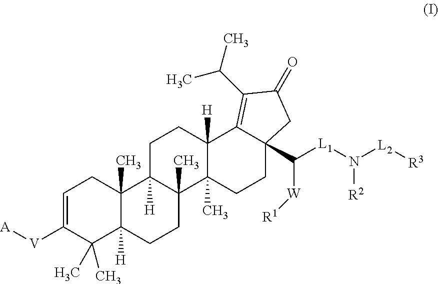

or a pharmaceutically acceptable salt thereof, wherein:

L₁ and L₂ are independently selected from a bond or [C(R⁶R^{6′})]_{q};

W is selected from a single bond or O;

R¹ is selected from the group consisting of —H, (C₁-C₁₂)alkyl, —C(O)R⁵, —CH₂—O—(C₁-C₆)alkyl, and 2-tetrahydro-2H-pyran;

R² is selected from the group consisting of —H, (C₁-C₁₂)alkyl, —(C₁-C₆)alkyl-OR⁴, —(C₁-C₆)alkyl-O—(C₁-C₆)alkyl, —C(O)R⁵, —(CH₂)₁NR⁷R⁸, and —(CH₂)₁N⁺(R⁴)₃, wherein when W is O, R¹ and R² can optionally be taken together with the O and N to which they are respectively joined to form a 4 to 8 membered heterocyclyl ring, wherein the heterocyclyl ring may be optionally substituted by one to two R¹¹ groups;

R³ is selected from the group consisting of hydrogen, (C₁-C₁₂)alkyl, —NR¹R², —OR⁵,

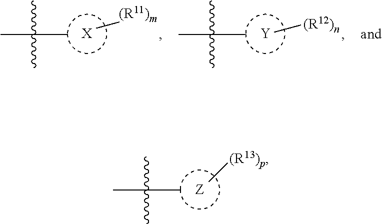

wherein:
X is a monocyclic or bicyclic (C₅-C₁₄)aryl,
Y is selected from a monocyclic or bicyclic (C₂-C₉)heterocyclyl or monocylic or bicyclic (C₂-C₉)heteroaryl, each having one to three heteroatoms selected from S, N or O, and
Z is a monocyclic or bicyclic (C₃-C₈)cycloalkyl;

R² and R³ can optionally be taken together with the nitrogen and L₂ to which they are respectively joined to form a 4 to 8 membered heterocyclyl ring, wherein the heterocyclyl ring may be optionally substituted by one to two R¹¹ groups;

R⁴ is selected from the group consisting of —H and (C₁-C₆)alkyl;

R⁵ is selected from the group consisting of —H, (C₁-C₆)alkyl, —R³, —(CH₂)₁NR⁷R⁸, and —(CH₂)ᵣOR⁷;

R⁶ and R^{6′} are independently selected from the group consisting of —H, (C₁-C₆)alkyl, (C₃-C₈)cycloalkyl, (C₁-C₆)alkoxy, haloalkyl, —Y, —(CH₂)₁NR⁷R⁸, —C(O)OH, and —C(O)NH₂, wherein the R⁶ and R^{6′} groups can optionally be taken together with the carbon to which they are joined to form a 3 to 8 membered cycloalkyl ring, and wherein the cycloalkyl ring may be optionally substituted by one to three R¹¹ groups;

R⁷ and R⁸ are independently selected from the group consisting of —H, (C₁-C₆)alkyl, (C₃-C₈)cycloalkyl, -Q-aryl-(R⁴)ₙ, —NR¹⁴R¹⁵, —C(O)CH₃, wherein R⁷ and R⁸ can optionally be taken together with the nitrogen to which they are joined to form a 4 to 8 membered heterocyclyl or heteroaryl ring containing one to three heteroatoms selected from —NR⁵—, —O—, —S—, —S(O)—, or —SO₂—, wherein the heterocyclyl or heteroaryl ring may be optionally substituted by one to three R¹¹ groups;

R⁹ is halo;

R¹⁰ is —N(R¹⁶)₂;

R¹¹, R¹², and R¹³ are independently selected from the group consisting of oxo, hydroxyl, halo, (C₁-C₆)alkoxy, —R⁶(R⁹)_{q}, —OR⁶(R⁹)_{q}, nitro, —SO₂R⁶, (C₁-C₆)alkyl, —C(O)R¹⁰, —R⁴YR⁶, —CO(O)R⁴, and —CO(O)R⁵, wherein any two R¹¹, R¹² or R¹³ groups can optionally join to form a 3 to 8 membered cycloalkyl, aryl, heterocyclyl or heteroaryl ring, wherein the heterocyclyl or heteroaryl ring may contain one to three heteroatoms selected from —NR⁵—, —O—, —S—, —S(O)—, or —SO₂—, and wherein the cycloalkyl, aryl, heterocyclyl or heteroaryl ring may be optionally substituted by one to three R¹⁶ groups;

R¹⁴ and R¹⁵ are independently selected from the group consisting of —H, (C₁-C₆)alkyl, (C₃-C₈)cycloalkyl, (C₁-C₆)alkoxy, —[C(R⁶)₂]ᵣ—, —O[C(R⁶)₂]ᵣ—, oxo, hydroxyl, halo, —C(O)R⁷, —R¹⁰, and —CO(O)R², wherein R¹⁴ and R¹⁵ can optionally be taken together with the carbon to which they are joined to form a 3 to 8 membered cycloalkyl ring or 4 to 8 membered heterocyclyl ring containing one to three heteroatoms selected from —NR⁵—, —O—, —S—, —S(O)—, or —SO₂—, wherein the cycloalkyl ring or heterocyclyl ring may be optionally substituted by one to three R¹⁶ groups;

R¹⁶ is selected from the group consisting of —H, halo, oxo, hydroxyl, (C₁-C₆)alkyl, (C₁-C₆)alkoxy, (C₃-C₈)cycloalkyl, —R⁶(R⁹)_{q}, —OR⁶(R⁹)_{q}, —N(R⁴)₂, —(CH₂)ᵣ-heterocycle, —C(O)OH, —C(O)NH₂, —R⁵(R⁹)_{q}, —OR⁵(R⁹)_{q}, nitro, —SO₂R⁶, —C(O)R¹⁰, and —CO(O)R⁴;

V is selected from the group consisting of —(C₄-C₈)cycloalkyl, —(C₄-C₈)cycloalkenyl, —(C₄-C₉)spirocycloalkyl, —(C₄-C₈)spirocycloalkenyl, —(C₄-C₈)oxacycloalkyl, —(C₄-C₈)oxacycloalkenyl, —(C₄-C₈)dioxacycloalkyl, —(C₄-C₈)dioxacycloalkenyl, —C₆ cyclodialkenyl, —C₆ oxacyclodialkenyl, —(C₆-C₉)oxaspirocycloalkyl, —(C₆-C₉)oxaspirocycloalkenyl,

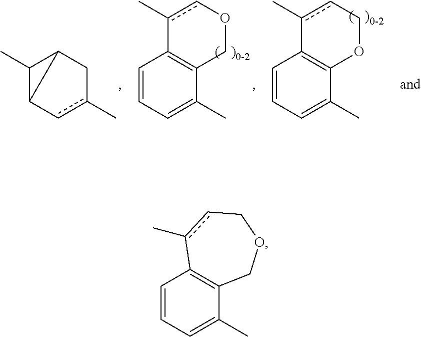

wherein:

V may be substituted with A², wherein:

A² is at least one member selected from the group consisting of —H, -halo, -hydroxyl, —(C₁-C₆)alkyl, —(C₁-C₆)alkoxy, —(C₁-C₆)alkyl-Q, -alkylsubstituted (C₁-C₆)alkyl-Q, —CN, —CF₂Q-NR¹⁷R¹⁷, —COOR¹⁷, and —CONR¹⁷R¹⁷, wherein:

Q is selected from the group consisting of aryl, heteroaryl, substituted heteroaryl, —OR¹⁷, —COOR¹⁸, —NR¹⁷R¹⁷, —SO₂R¹⁹, —CONHSO₂R¹⁸, and —CONHSO₂NR¹⁷R¹⁷;

R¹⁷ is selected from the group consisting of —H, —(C₁-C₆)alkyl, -alkylsubstituted (C₁-C₆)alkyl, and -arylsubstituted (C₁-C₆)alkyl;

R¹⁸ is selected from the group consisting of —(C₁-C₆)alkyl and -alkylsubstituted (C₁-C₆)alkyl;

R¹⁹ is selected from the group consisting of —(C₁-C₆)alkyl, —(C₁-C₆)substituted alkyl, —(C₃-C₆)cycloalkyl, —CF₃, aryl, and heteroaryl;

A is selected from the group consisting of —COOR¹⁷, —C(O)NR¹⁷SO₂R¹⁸, —C(O)NHSO₂NR¹⁷R¹⁷, —NR¹⁷SO₂R¹⁷, —SO₂NR¹⁷R¹⁷, —(C₃-C₆)cycloalkyl-COOR¹⁷, —(C₂-C₆)alkenyl-COOR¹⁷, —(C₂-C₆)alkynyl-COOR¹⁷, —(C₁-C₆)alkyl-COOR¹⁷, -alkylsubstituted (C₁-C₆)alkyl, —CF₂—COOR¹⁷, —NHC(O)(CH₂)ₙ₁—COOR¹⁷, —SO₂NR¹⁷C(O)R¹⁷, tetrazole, and —C(O)NHOH,

m and n in each instance are independently 0, 1, 2, 3, or 4;

p is independently 0, 1, 2, 3, or 4;

r and q in each instance are independently 0, 1, 2, 3, or 4; and

n¹ is independently 1, 2, 3, 4, 5, or 6.

In a second aspect, the present invention relates to a pharmaceutical composition comprising a) the compound of Formula I or a pharmaceutically acceptable salt the thereof; and b) a pharmaceutically acceptable excipient.

In a third aspect, the present invention is a method of treating an HIV infection comprising administering to a subject suffering there from a compound of Formula I, or a pharmaceutically acceptable salt thereof.

Compounds of the present invention are useful for the treatment of subjects with an HIV infection or for the treatment of subjects at risk of acquiring an HIV infection.

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

As used herein unless otherwise specified, “alkyl” refers to a monovalent saturated aliphatic hydrocarbyl group having from 1 to 14 carbon atoms and, in some embodiments, from 1 to 6 carbon atoms. “(Cₓ-C_{y})alkyl” refers to alkyl groups having from x to y carbon atoms. The term “alkyl” includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkylene” or “alkylene” refers to divalent saturated aliphatic hydrocarbyl groups having from 1 to 10 carbon atoms and, in some embodiments, from 1 to 6 carbon atoms. “(Cᵤ-Cᵥ)alkylene” refers to alkylene groups having from u to v carbon atoms. The alkylene groups include branched and straight chain hydrocarbyl groups. For example, “(C₁-C₆)alkylene” is meant to include methylene, ethylene, propylene, 2-methypropylene, dimethylethylene, pentylene, and so forth. As such, the term “propylene” could be exemplified by the following structure:

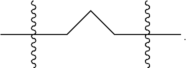 [SMILES: *CCC*]

Likewise, the term “dimethylbutylene” could be exemplified by any of the following three structures or more:

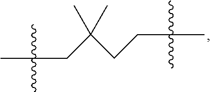 [SMILES: *CCC(C)(C)C*]

p, or

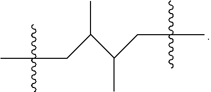 [SMILES: *CC(C)C(C)C*]

Furthermore, the term “(C₁-C₆)alkylene” is meant to include such branched chain hydrocarbyl groups as cyclopropylmethylene, which could be exemplified by the following structure:

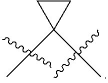 [SMILES: *C1(*)CC1]

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, isopropylene, 1,3-butadienyl, and the like.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Acyl” refers to the groups H—C(O)—, alkyl-C(O)—, alkenyl-C(O)—, alkynyl-C(O)—, cycloalkyl-C(O)—, aryl-C(O)—, heteroaryl-C(O)—, and heterocyclic-C(O)—. Acyl includes the “acetyl” group CH₃C(O)—.

“Acylamino” refers to the

groups —NR²⁰C(O)alkyl, —NR²⁰C(O)cycloalkyl, —NR²⁰C(O)alkenyl, —NR²⁰C(O)alkynyl, —NR²⁰C(O)aryl, —NR²⁰C(O)heteroaryl, and —NR²⁰C(O)heterocyclic, wherein R²⁰ is hydrogen or alkyl.

“Acyloxy” refers to the groups alkyl-C(O)O—, alkenyl-C(O)O—, alkynyl-C(O)O—, aryl-C(O)O—, cycloalkyl-C(O)O—, heteroaryl-C(O)O—, and heterocyclic-C(O)O—.

“Amino” refers to the group —NR²¹R²² where R²¹ and R²² are independently selected from hydrogen, alkyl, alkenyl, alkynyl, aryl, cycloalkyl, heteroaryl, heterocyclic, —SO₂-alkyl, —SO₂-alkenyl, —SO₂-cycloalkyl, —SO₂-aryl, —SO₂-heteroaryl, and —SO₂-heterocyclic, and wherein R²¹ and R²² are optionally joined together with the nitrogen bound thereto to form a heterocyclic group. When R²¹ is hydrogen and R²² is alkyl, the amino group is sometimes referred to herein as alkylamino. When R²¹ and R²² are alkyl, the amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²⁶R²⁷ where R²⁶ and R²⁷ are independently selected from hydrogen, alkyl, alkenyl, alkynyl, aryl, cycloalkyl, heteroaryl, heterocyclic, hydroxy, alkoxy, amino, and acylamino, and where R²⁶ and R²⁷ are optionally joined together with the nitrogen bound thereto to form a heterocyclic group.

“Aryl” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“AUC” refers to the area under the plot of plasma concentration of drug (not logarithm of the concentration) against time after drug administration.

“EC₅₀” refers to the concentration of a drug that gives half-maximal response.

“IC₅₀” refers to the half-maximal inhibitory concentration of a drug. Sometimes, it is also converted to the pIC₅₀ scale (−log IC₅₀), in which higher values indicate exponentially greater potency.

“Clade” refers to a hypothetical construct based on experimental data. Clades are found using multiple (sometimes hundreds) of traits from a number of species (or specimens) and analyzing them statistically to find the most likely phylogenetic tree for the group.

“Cyano” or “nitrile” refers to the group —CN.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups, such as cyclohexenyl. Examples of cycloalkyl groups include, for instance, adamantyl, cyclopropyl, cyclobutyl, cyclohexyl, cyclopentyl, cyclooctyl, cyclopentenyl, and cyclohexenyl. Examples of cycloalkyl groups that include multiple bicycloalkyl ring systems are bicyclohexyl, bicyclopentyl, bicyclooctyl, and the like. Two such bicycloalkyl multiple ring structures are exemplified and named below:

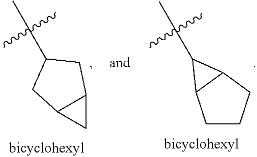 [SMILES: *C1CC2CC2C1 ; *C1C2CCCC12]

“(Cᵤ-Cᵥ)cycloalkyl” refers to cycloalkyl groups having u to v carbon atoms.

“Spiro cycloalkyl” refers to a 3 to 10 member cyclic substituent formed by replacement of two hydrogen atoms at a common carbon atom in a cyclic ring structure or in an alkylene group having 2 to 9 carbon atoms, as exemplified by the following structure wherein the group shown here attached to bonds marked with wavy lines is substituted with a spiro cycloalkyl group:

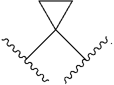 [SMILES: *C1(*)CC1]

“Fused cycloalkyl” refers to a 3 to 10 member cyclic substituent formed by the replacement of two hydrogen atoms at different carbon atoms in a cycloalkyl ring structure, as exemplified by the following structure wherein the cycloalkyl group shown here contains bonds marked with wavy lines which are bonded to carbon atoms that are substituted with a fused cycloalkyl group:

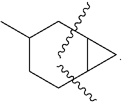

“Carboxy” or “carboxyl” refers interchangeably to the groups

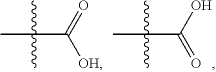

### —C(O)O, or —CO₂.

“Halo” or “halogen” refers to fluoro, chloro, bromo, and iodo.

“Haloalkyl” refers to substitution of an alkyl group with 1 to 3 halo groups (e.g., bifluoromethyl or trifluoromethyl).

“Haloalkoxy” refers to substitution of alkoxy groups with 1 to 5 (e.g. when the alkoxy group has at least 2 carbon atoms) or in some embodiments 1 to 3 halo groups (e.g. trifluoromethoxy).

“Human Serum Protein Shift Assay” refers to an HIV assay using a Luciferase Reporter to determine percent inhibition—pIC₅₀. The HIV assay makes use of a two-cell co-culture system. In this assay, an infected cell line J4HxB2 and an indicator cell line HOS (delta LTR+luciferase) are co-cultured in the presence and absence of compound. The assay is designed to find inhibitors that prevent the infection of HOS cells by the J4HxB2 cell line. The assay can detect inhibitors of any stage of the HIV infection cycle.

“Hydroxy” or “hydroxyl” refers to the group —OH.

“Heteroaryl” refers to an aromatic group of from 1 to 14 carbon atoms and 1 to 6 heteroatoms selected from oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, imidazolinyl, isoxazolyl, pyrrolyl, pyrazolyl, pyridazinyl, pyrimidinyl, purinyl, phthalazyl, naphthylpryidyl, benzofuranyl, tetrahydrobenzofuranyl, isobenzofuranyl, benzothiazolyl, benzoisothiazolyl, benzotriazolyl, indolyl, isoindolyl, indolizinyl, dihydroindolyl, indazolyl, indolinyl, benzoxazolyl, quinolyl, isoquinolyl, quinolizyl, quianazolyl, quinoxalyl, tetrahydroquinolinyl, isoquinolyl, quinazolinonyl, benzimidazolyl, benzisoxazolyl, benzothienyl, benzopyridazinyl, pteridinyl, carbazolyl, carbolinyl, phenanthridinyl, acridinyl, phenanthrolinyl, phenazinyl, phenoxazinyl, phenothiazinyl, and phthalimidyl.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from nitrogen, sulfur, phosphorus or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen, phosphorus and/or sulfur atom(s) of the heterocyclic group are optionally oxidized to provide for the N-oxide, phosphinane oxide, sulfinyl, sulfonyl moieties. More specifically the heterocyclyl includes, but is not limited to, tetrahydropyranyl, piperidinyl, piperazinyl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

Examples of heterocycle and heteroaryl groups include, but are not limited to, azetidine, pyrrole, imidazole, pyrazole, pyridine, pyrazine, pyrimidine, pyridazine, pyridone, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholine, thiomorpholine (also referred to as thiamorpholine), piperidine, pyrrolidine, and tetrahydrofuranyl.

“Fused heterocyclic” or “fused heterocycle” refer to a 3 to 10 member cyclic substituent formed by the replacement of two hydrogen atoms at different carbon atoms in a cycloalkyl ring structure, as exemplified by the following structure wherein the cycloalkyl group shown here contains bonds marked with wavy lines which are bonded to carbon atoms that are substituted with a fused heterocyclic group:

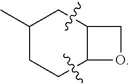

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

The term “heteroatom” means nitrogen, oxygen, or sulfur and includes any oxidized form of nitrogen, such as N(O) {N⁺—O⁻} and sulfur such as S(O) and S(O)₂, and the quaternized form of any basic nitrogen.

“Oxazolidinone” refers to a 5-membered heterocyclic ring containing one nitrogen and one oxygen as heteroatoms and also contains two carbons and is substituted at one of the two carbons by a carbonyl group as exemplified by any of the following structures, wherein the oxazolidinone groups shown here are bonded to a parent molecule, which is indicated by a wavy line in the bond to the parent molecule:

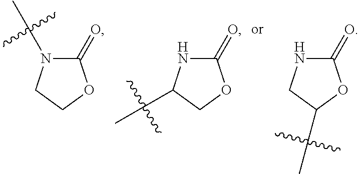 [SMILES: *N1CCOC1=O ; *C1COC(=O)N1 ; *C1CNC(=O)O1]

“Oxo” refers to a (═O) group.

“Polymorphism” refers to when two or more clearly different phenotypes exist in the same population of a species where the occurrence of more than one form or morph. In order to be classified as such, morphs must occupy the same habitat at the same time and belong to a panmictic population (one with random mating).

“Protein binding” refers to the binding of a drug to proteins in blood plasma, tissue membranes, red blood cells and other components of blood.

“Protein shift” refers to determining a binding shift by comparing the EC₅₀ values determined in the absence and presence of human serum.

“QVT” refers to the amino acids at positions 369, 370, and 371, respectively in the Sp1 fragment of HIV-1 Gag.

“Racemates” refers to a mixture of enantiomers. In an embodiment of the invention, the compounds of Formula I, or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

The term ‘atropisomer’ refers to a stereoisomer resulting from an axis of asymmetry. This can result from restricted rotation about a single bond where the rotational barrier is high enough to allow differentiation of the isomeric species up to and including complete isolation of stable non-interconverting diastereomer or enantiomeric species. One skilled in the art will recognize that upon installing a nonsymmetrical R^{x} to core, the formation of atropisomers is possible. In addition, once a second chiral center is installed in a given molecule containing an atropisomer, the two chiral elements taken together can create diastereomeric and enantiomeric stereochemical species. Depending upon the substitution about the Cx axis, interconversion between the atropisomers may or may not be possible and may depend on temperature. In some instances, the atropisomers may interconvert rapidly at room temperature and not resolve under ambient conditions. Other situations may allow for resolution and isolation but interconversion can occur over a period of seconds to hours or even days or months such that optical purity is degraded measurably over time. Yet other species may be completely restricted from interconversion under ambient and/or elevated temperatures such that resolution and isolation is possible and yields stable species. When known, the resolved atropisomers were named using the helical nomenclature. For this designation, only the two ligands of highest priority in front and behind the axis are considered. When the turn priority from the front ligand 1 to the rear ligand 1 is clockwise, the configuration is P, if counterclockwise it is M.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” or “subject” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Wherever dashed lines occur adjacent to single bonds denoted by solid lines, then the dashed line represents an optional double bond at that position. Likewise, wherever dashed circles appear within ring structures denoted by solid lines or solid circles, then the dashed circles represent one to three optional double bonds arranged according to their proper valence taking into account whether the ring has any optional substitutions around the ring as will be known by one of skill in the art. For example, the dashed line in the structure below could either indicate a double bond at that position or a single bond at that position:

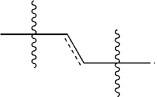

Similarly, ring A below could be a cyclohexyl ring without any double bonds or it could also be a phenyl ring having three double bonds arranged in any position that still depicts the proper valence for a phenyl ring. Likewise, in ring B below, any of X¹-X⁵ could be selected from: C, CH, or CH₂, N, or NH, and the dashed circle means that ring B could be a cyclohexyl or phenyl ring or a N-containing heterocycle with no double bonds or a N-containing heteroaryl ring with one to three double bonds arranged in any position that still depicts the proper valence:

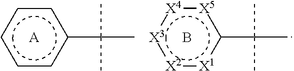

Where specific compounds or generic formulas are drawn that have aromatic rings, such as aryl or heteroaryl rings, then it will understood by one of still in the art that the particular aromatic location of any double bonds are a blend of equivalent positions even if they are drawn in different locations from compound to compound or from formula to formula. For example, in the two pyridine rings (A and B) below, the double bonds are drawn in different locations, however, they are known to be the same structure and compound:

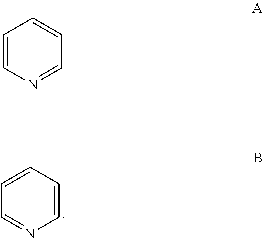 [SMILES: c1ccncc1 ; c1ccncc1]

The present invention includes compounds as well as their pharmaceutically acceptable salts. Accordingly, the word “or” in the context of “a compound or a pharmaceutically acceptable salt thereof” is understood to refer to either: 1) a compound alone or a compound and a pharmaceutically acceptable salt thereof (alternative), or 2) a compound and a pharmaceutically acceptable salt thereof (in combination).

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—. In a term such as “—C(R^{x})₂”, it should be understood that the two R^{x} groups can be the same, or they can be different if R^{x} is defined as having more than one possible identity. In addition, certain substituents are drawn as —R^{x}R^{y}, where the “—” indicates a bond adjacent to the parent molecule and R^{y} being the terminal portion of the functionality. Similarly, it is understood that the above definitions are not intended to include impermissible substitution patterns (e.g., methyl substituted with 5 fluoro groups). Such impermissible substitution patterns are well known to the skilled artisan.

As recited above, Bevirimat is a yet unapproved anti-HIV drug derived from a betulinic acid-like compound, first isolated from Syzygium claviflorum, a Chinese herb. It is believed to inhibit HIV by a novel mechanism, so-called maturation inhibition. Like protease inhibitors, Bevirimat and other maturation inhibitors interfere with protease processing of newly translated HIV polyprotein precursor, called gag. Gag is an essential structural protein of the HIV virus. Gag undergoes a chain of interactions both with itself and with other cellular and viral factors to accomplish the assembly of infectious virus particles.

However, naturally occurring polymorphisms in HIV are present in some infected individuals, thus lowering the anti-HIV efficacy of some currently considered therapies. Indeed, studies have shown that presence of a number of single nucleotide polymorphisms in the Capsid/SP1 spacer protein (CA/SP1) cleavage site has resulted in clinical resistance in HIV patients to Bevirimat. Likewise, mutations in the glutamine-valine-threonine (QVT) motif of the SP1 peptide are also known to cause Bevirimat resistance in HIV infected patients. Mutations in the QVT motif of the SP1 peptide are the primary predictors of failure to respond to Bevirimat and the effect of these mutations has been repeatedly demonstrated. These problems eventually led to the cessation of clinical development of Bevirimat. See Knapp, D., et al., J. Clin. Microbiol. 49(1): 201-208 (2011). See previously filed WO 2013/090664 for Bevirimat data.

In accordance with one embodiment of the present invention, there is provided a compound having the structure of Formula I:

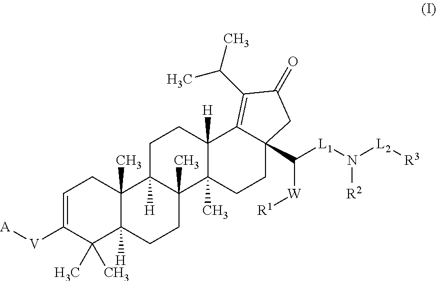

or a pharmaceutically acceptable salt thereof, wherein:

L₁ and L₂ are independently selected from a bond or [C(R⁶R^{6′})]_{q};

W is selected from a single bond or O;

R¹ is selected from the group consisting of —H, (C₁-C₁₂)alkyl, —C(O)R⁵, —CH₂—O—(C₁-C₆)alkyl, and 2-tetrahydro-2H-pyran;

R² is selected from the group consisting of —H, (C₁-C₁₂)alkyl, —(C₁-C₆)alkyl-OR⁴, —(C₁-C₆)alkyl-O—(C₁-C₆)alkyl, —C(O)R⁵, —(CH₂)₁NR⁷R⁸, and —(CH₂)₁N⁺(R⁴)₃, wherein when W is O, R¹ and R² can optionally be taken together with the O and N to which they are respectively joined to form a 4 to 8 membered heterocyclyl ring, wherein the heterocyclyl ring may be optionally substituted by one to two R¹¹ groups;

R³ is selected from the group consisting of hydrogen, (C₁-C₁₂)alkyl, —NR¹R², —OR⁵,

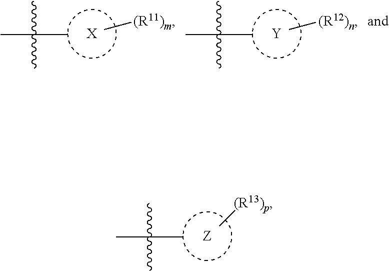

wherein:
X is a monocyclic or bicyclic (C₅-C₁₄)aryl,
Y is selected from a monocyclic or bicyclic (C₂-C₉)heterocyclyl or monocylic or bicyclic (C₂-C₉)heteroaryl, each having one to three heteroatoms selected from S, N or O, and
Z is a monocyclic or bicyclic (C₃-C₈)cycloalkyl;

R² and R³ can optionally be taken together with the nitrogen and L₂ to which they are respectively joined to form a 4 to 8 membered heterocyclyl ring, wherein the heterocyclyl ring may be optionally substituted by one to two R¹¹ groups;

R⁴ is selected from the group consisting of —H and (C₁-C₆)alkyl;

R⁵ is selected from the group consisting of —H, (C₁-C₆)alkyl, —R³, —(CH₂)₁NR⁷R⁸, and —(CH₂)ᵣOR⁷;

R⁶ and R^{6′} are independently selected from the group consisting of —H, (C₁-C₆)alkyl, (C₃-C₈)cycloalkyl, (C₁-C₆)alkoxy, haloalkyl, —Y, —(CH₂)₁NR⁷R⁸, —C(O)OH, and —C(O)NH₂, wherein the R⁶ and R^{6′} groups can optionally be taken together with the carbon to which they are joined to form a 3 to 8 membered cycloalkyl ring, and wherein the cycloalkyl ring may be optionally substituted by one to three R¹¹ groups;

R⁷ and R⁸ are independently selected from the group consisting of —H, (C₁-C₆)alkyl, (C₃-C₈)cycloalkyl, -Q-aryl-(R⁴)ₙ, —NR¹⁴R¹⁵, —C(O)CH₃, wherein R⁷ and R⁸ can optionally be taken together with the nitrogen to which they are joined to form a 4 to 8 membered heterocyclyl or heteroaryl ring containing one to three heteroatoms selected from —NR⁵—, —O—, —S—, —S(O)—, or —SO₂—, wherein the heterocyclyl or heteroaryl ring may be optionally substituted by one to three R¹¹ groups;

R⁹ is halo;

R¹⁰ is —N(R¹⁶)₂;

R¹¹, R¹², and R¹³ are independently selected from the group consisting of oxo, hydroxyl, halo, (C₁-C₆)alkoxy, —R⁶(R⁹)_{q}, —OR⁶(R⁹)_{q}, nitro, —SO₂R⁶, (C₁-C₆)alkyl, —C(O)R¹⁰, —R⁴YR⁶, —CO(O)R⁴, and —CO(O)R⁵, wherein any two R¹¹, R¹² or R¹³ groups can optionally join to form a 3 to 8 membered cycloalkyl, aryl, heterocyclyl or heteroaryl ring, wherein the heterocyclyl or heteroaryl ring may contain one to three heteroatoms selected from —NR⁵—, —O—, —S—, —S(O)—, or —SO₂—, and wherein the cycloalkyl, aryl, heterocyclyl or heteroaryl ring may be optionally substituted by one to three R¹⁶ groups;

R¹⁴ and R¹⁵ are independently selected from the group consisting of —H, (C₁-C₆)alkyl, (C₃-C₈)cycloalkyl, (C₁-C₆)alkoxy, —[C(R⁶)₂]ᵣ—, —O[C(R⁶)₂]ᵣ—, oxo, hydroxyl, halo, —C(O)R⁷, —R¹⁰, and —CO(O)R², wherein R¹⁴ and R¹⁵ can optionally be taken together with the carbon to which they are joined to form a 3 to 8 membered cycloalkyl ring or 4 to 8 membered heterocyclyl ring containing one to three heteroatoms selected from —NR⁵—, —O—, -5-, —S(O)—, or —SO₂—, wherein the cycloalkyl ring or heterocyclyl ring may be optionally substituted by one to three R¹⁶ groups;

R¹⁶ is selected from the group consisting of —H, halo, oxo, hydroxyl, (C₁-C₆)alkyl, (C₁-C₆)alkoxy, (C₃-C₈)cycloalkyl, —R⁶(R⁹)_{q}, —OR⁶(R⁹)_{q}, —N(R⁴)₂, —(CH₂)ᵣ-heterocycle, —C(O)OH, —C(O)NH₂, —R⁵(R⁹)_{q}, —OR⁵(R⁹)_{q}, nitro, —SO₂R⁶, —C(O)R¹⁰, and —CO(O)R⁴;

V is selected from the group consisting of —(C₄-C₈)cycloalkyl, —(C₄-C₈)cycloalkenyl, —(C₄-C₉)spirocycloalkyl, —(C₄-C₈)spirocycloalkenyl, —(C₄-C₈)oxacycloalkyl, —(C₄-C₈)oxacycloalkenyl, —(C₄-C₈)dioxacycloalkyl, —(C₄-C₈)dioxacycloalkenyl, —C₆ cyclodialkenyl, —C₆ oxacyclodialkenyl, —(C₆-C₉)oxaspirocycloalkyl, —(C₆-C₉)oxaspirocycloalkenyl,

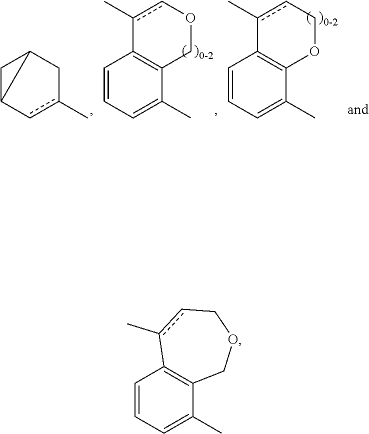

wherein:

V may be substituted with A², wherein:

A² is at least one member selected from the group consisting of —H, -halo, -hydroxyl, —(C₁-C₆)alkyl, —(C₁-C₆)alkoxy, —(C₁-C₆)alkyl-Q, -alkylsubstituted (C₁-C₆)alkyl-Q, —CN, —CF₂Q-NR¹⁷R¹⁷, —COOR¹⁷, and —CONR¹⁷R¹⁷, wherein:

Q is selected from the group consisting of aryl, heteroaryl, substituted heteroaryl, —OR¹⁷, —COOR¹⁸, —NR¹⁷R¹⁷, —SO₂R¹⁹, —CONHSO₂R¹⁸, and —CONHSO₂NR¹⁷R¹⁷;

R¹⁷ is selected from the group consisting of —H, —(C₁-C₆)alkyl, -alkylsubstituted (C₁-C₆)alkyl, and -arylsubstituted (C₁-C₆)alkyl;

R¹⁸ is selected from the group consisting of —(C₁-C₆)alkyl and -alkylsubstituted (C₁-C₆)alkyl;

R¹⁹ is selected from the group consisting of —(C₁-C₆)alkyl, —(C₁-C₆)substituted alkyl, —(C₃-C₆)cycloalkyl, —CF₃, aryl, and heteroaryl;

A is selected from the group consisting of —COOR¹⁷, —C(O)NR¹⁷SO₂R¹⁸, —C(O)NHSO₂NR¹⁷R¹⁷, —NR¹⁷SO₂R¹⁷, —SO₂NR¹⁷R¹⁷, —(C₃-C₆)cycloalkyl-COOR¹⁷, —(C₂-C₆)alkenyl-COOR¹⁷, —(C₂-C₆)alkynyl-COOR¹⁷, —(C₁-C₆)alkyl-COOR¹⁷, -alkylsubstituted (C₁-C₆)alkyl, —CF₂—COOR¹⁷, —NHC(O)(CH₂)ₙ₁—COOR¹⁷, —SO₂NR¹⁷C(O)R¹⁷, tetrazole, and —C(O)NHOH,

m and n in each instance are independently 0, 1, 2, 3, or 4;

p is independently 0, 1, 2, 3, or 4;

r and q in each instance are independently 0, 1, 2, 3, or 4; and

n¹ is independently 1, 2, 3, 4, 5, or 6.

In accordance with another embodiment of the present invention, there is provided a compound having the structure of Formula I:

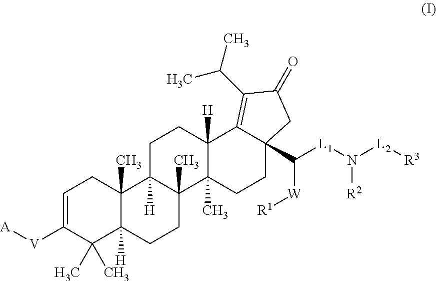

or a pharmaceutically acceptable salt thereof, wherein:

L₁ and L₂ are [C(R⁶R^{6′})]_{q};

W is selected from a single bond or O;

R¹ is selected from the group consisting of —H, (C₁-C₆)alkyl, and —C(O)R⁴;

R² is selected from the group consisting of —H, (C₁-C₆)alkyl, —(C₁-C₆)alkyl-OR⁴, —(C₁-C₆)alkyl-O—(C₁-C₆)alkyl, —C(O)R⁵, —(CH₂)₁NR⁷R⁸, and —(CH₂)₁N⁺(R⁴)₃;

R³ is selected from the group consisting of —H, (C₁-C₁₂)alkyl, —NR¹R², —OR⁵,

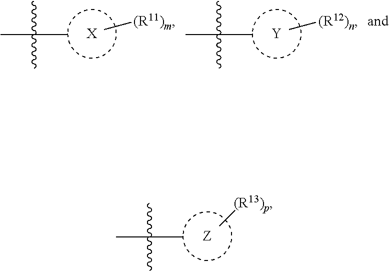

wherein:
X is a monocyclic or bicyclic (C₅-C₁₄)aryl,
Y is selected from a monocyclic or bicyclic (C₂-C₉)heterocyclyl or monocylic or bicyclic (C₂-C₉)heteroaryl, each having one to three heteroatoms selected from S, N or O, and
Z is a monocyclic or bicyclic (C₃-C₈)cycloalkyl;

R⁴ is selected from the group consisting of —H and (C₁-C₆)alkyl;

R⁵ is selected from the group consisting of (C₁-C₆)alkyl, —(CH₂)₁NR⁷R⁸, and —(CH₂)ᵣOR⁷;

R⁶ and R^{6′} are independently selected from the group consisting of —H, (C₁-C₆)alkyl, (C₃-C₈)cycloalkyl, (C₁-C₆)alkoxy, haloalkyl, —(CH₂)₁NR⁷R⁸, —C(O)OH, and —C(O)NH₂, wherein the R⁶ and R^{6′} groups can optionally be taken together with the carbon to which they are joined to form a 3 to 8 membered cycloalkyl ring, and wherein the cycloalkyl ring may be optionally substituted by one to three R¹¹ groups;

R⁷ and R⁸ are independently selected from the group consisting of —H, (C₁-C₆)alkyl, (C₃-C₈)cycloalkyl, —NR¹⁴R¹⁵, and —C(O)CH₃, wherein R⁷ and R⁸ can optionally be taken together with the nitrogen to which they are joined to form a 4 to 8 membered heterocyclyl or heteroaryl ring containing one to three heteroatoms selected from —NR⁵−, −O−, —S—, —S(O)—, or —SO₂—, wherein the heterocyclyl or heteroaryl ring may be optionally substituted by one to three R¹¹ groups;

R⁹ is halo;

R¹⁰ is —N(R¹⁶)₂;

R¹¹, R¹², and R¹³ are independently selected from the group consisting of oxo, hydroxyl, halo, (C₁-C₆)alkoxy, —R⁶(R⁹)_{q}, —OR⁶(R⁹)_{q}, nitro, —SO₂R⁶, (C₁-C₆)alkyl, —C(O)R¹⁰, —R⁴YR⁶, —CO(O)R⁴, and —CO(O)R⁵;

R¹⁴ and R¹⁵ are independently selected from the group consisting of —H, (C₁-C₆)alkyl, (C₃-C₈)cycloalkyl, (C₁-C₆)alkoxy, —[C(R⁶)₂]ᵣ—, —O[C(R⁶)₂]ᵣ—, oxo, hydroxyl, halo, —C(O)R⁷, —R¹⁰, and —CO(O)R²;

R¹⁶ is independently selected from the group consisting of —H, oxo, halo, hydroxyl, (C₁-C₆)alkyl, (C₁-C₆)alkoxy, (C₃-C₈)cycloalkyl, —R⁶(R⁹)_{q}, —OR⁶(R⁹)_{q}, —N(R⁴)₂, —(CH₂)ᵣ-heterocycle, —C(O)OH, —C(O)NH₂, —R⁵(R⁹)_{q}, —OR⁵(R⁹)_{q}, nitro, —SO₂R⁶, —C(O)R¹⁰, and —CO(O)R⁴;

m and n in each instance are independently 0, 1, 2, 3, or 4;

p is independently 0, 1, 2, 3, or 4; and

r and q in each instance are independently 0, 1, 2, 3, or 4.

In accordance with another embodiment of the present invention, there is provided a compound of Formula I above, wherein V is selected from a group consisting of —(C₄-C₈)cycloalkenyl, —(C₄-C₉)spirocycloalkyl, and —(C₄-C₉)spirocycloalkenyl.

In accordance with another embodiment of the present invention, there is provided a compound of Formula I above, wherein A is in the para position.

In accordance with another embodiment of the present invention, there is provided a compound of Formula I above, wherein A² is at least one member selected from the group the group consisting of —H, -halo, -hydroxyl, —(C₁-C₃)alkyl, —(C₁-C₃)alkoxy.

In accordance with another embodiment of the present invention, there is provided a compound of Formula I above, wherein A² is at least one member selected from the group group consisting of —H, —Cl, —F, and —Br.

In accordance with another embodiment of the present invention, there is provided a compound of Formula I above, wherein A² is at least one member selected from the group consisting of —F and —H.

In accordance with another embodiment of the present invention, there is provided a compound of Formula I above, wherein A² is —H.

In accordance with another embodiment of the present invention, there is provided a compound of Formula I above, wherein A is COOR¹⁷.

In accordance with another embodiment of the present invention, there is provided a compound of Formula I above, wherein A is —COOH.

In accordance with another embodiment of the present invention, there is provided a compound of Formula I above, wherein

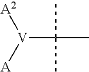

has the structure selected from the group consisting of the following:

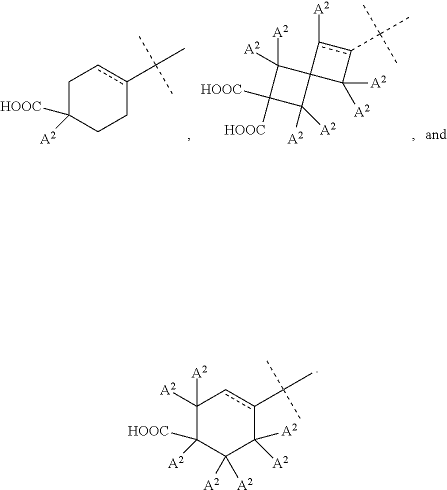

In accordance with another embodiment of the present invention, there is provided a compound of Formula I above, wherein

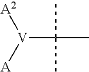

has the structure selected from the group consisting of the following:

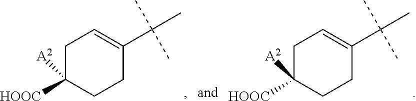

In accordance with another embodiment of the present invention, there is provided a compound of Formula I above, wherein

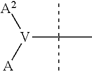

has the structure selected from the group consisting of the following:

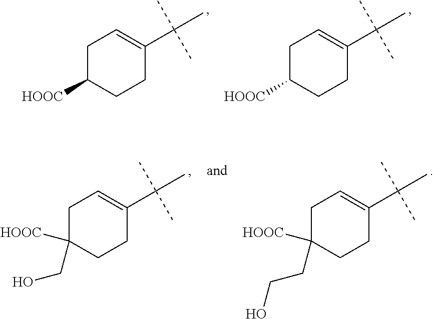

In accordance with another embodiment of the present invention, there is provided a compound having the structure of Formula I:

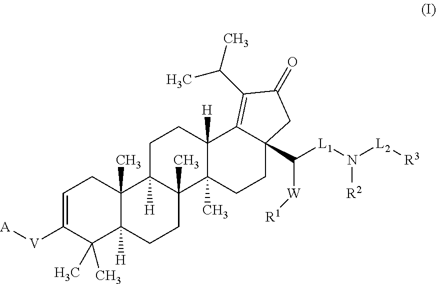

or a pharmaceutically acceptable salt thereof, wherein:

L₁ and L₂ are both (—CH₂—);

W is O;

R¹ is —H;

R² is selected from the group consisting of —H, (C₁-C₆)alkyl, —C(O)R⁵, and —(CH₂)ᵣNR⁷R⁸;

R³ is selected from the group consisting of

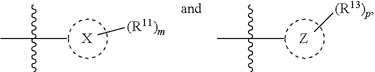

wherein:

X is a monocyclic or bicyclic (C₅-C₁₄)aryl,

Z is a monocyclic or bicyclic (C₃-C₈)cycloalkyl;

R⁴ is selected from the group consisting of —H and (C₁-C₆)alkyl;

R⁵ is selected from the group consisting of (C₁-C₆)alkyl, —(CH₂)₁NR⁷R⁸, and —(CH₂)ᵣOR⁷;

R⁶ is selected from the group consisting of —H, (C₁-C₆)alkyl, (C₃-C₈)cycloalkyl, (C₁-C₆)alkoxy, haloalkyl, —(CH₂)₁NR⁷R⁸, —C(O)OH, and —C(O)NH₂;

R⁷ and R⁸ are independently selected from the group consisting of —H, (C₁-C₆)alkyl, (C₃-C₈)cycloalkyl, —NR¹⁴R¹⁵, and —C(O)CH₃, wherein R⁷ and R⁸ can be taken together with the nitrogen to which they are joined to form a 4 to 8 membered heterocycle or heteroaryl ring containing one to three heteroatoms selected from —NR⁵, —O—, —S—, —S(O)—, or —SO2-;

R⁹ is halo;

R¹⁰ is —N(R¹⁶)₂;

R¹¹ and R¹³ are independently selected from the group consisting of oxo, hydroxyl, halo, (C₁-C₆)alkoxy, —R⁶(R⁹)_{q}, —OR⁶(R⁹)_{q}, nitro, —SO₂R⁶, (C₁-C₆)alkyl, —C(O)R¹⁰, —CO(O)R⁴, and —CO(O)R⁵;

R¹⁴ and R¹⁵ are independently selected from the group consisting of —H, (C₁-C₆)alkyl, (C₃-C₈)cycloalkyl, (C₁-C₆)alkoxy, —[C(R⁶)₂]ᵣ—, —O[C(R⁶)₂]ᵣ—, oxo, hydroxyl, halo, —C(O)R⁷, —R¹⁰, and —CO(O)R²;

R¹⁶ is selected from the group consisting of —H, oxo, halo, hydroxyl, (C₁-C₆)alkyl, (C₁-C₆)alkoxy, (C₃-C₈)cycloalkyl, —R⁶(R⁹)_{q}, —OR⁶(R⁹)_{q}, —N(R⁴)₂, —(CH₂)ᵣ-heterocycle, —C(O)OH, —C(O)NH₂, —R⁵(R⁹)_{q}, —OR⁵(R⁹)_{q}, nitro, —SO₂R⁶, —C(O)R¹⁰, and —CO(O)R⁴;

m and p in each instance are independently 0, 1, or 2;

r and q in each instance are independently 0, 1, 2, or 3.

In accordance with another embodiment of the present invention, there is provided a compound of Formula I above, wherein V is selected from a group consisting of —(C₄-C₈)cycloalkenyl, —(C₄-C₉)spirocycloalkyl, and —(C₄-C₉)spirocycloalkenyl.

In accordance with another embodiment of the present invention, there is provided a compound of Formula I above, wherein A is in the para position.

In accordance with another embodiment of the present invention, there is provided a compound of Formula I above, wherein A² is at least one member selected from the group the group consisting of —H, -halo, -hydroxyl, —(C₁-C₃)alkyl, —(C₁-C₃)alkoxy.

In accordance with another embodiment of the present invention, there is provided a compound of Formula I above, wherein A² is at least one member selected from the group group consisting of —H, —Cl, —F, and —Br.

In accordance with another embodiment of the present invention, there is provided a compound of Formula I above, wherein A² is at least one member selected from the group consisting of —F and —H.

In accordance with another embodiment of the present invention, there is provided a compound of Formula I above, wherein A² is —H.

In accordance with another embodiment of the present invention, there is provided a compound of Formula I above, wherein A is COOR¹⁷.

In accordance with another embodiment of the present invention, there is provided a compound of Formula I above, wherein A is —COOH.

In accordance with another embodiment of the present invention, there is provided a compound of Formula I above, wherein

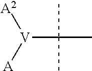

has the structure selected from the group consisting of the following:

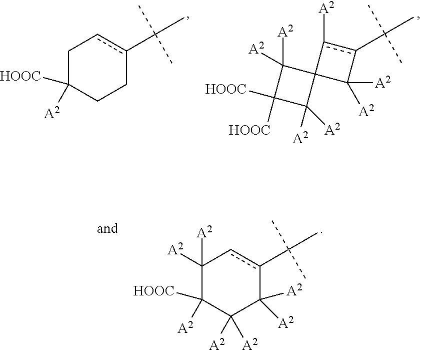

In accordance with another embodiment of the present invention, there is provided a compound of Formula I above, wherein

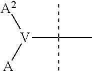

has the structure selected from the group consisting of the following:

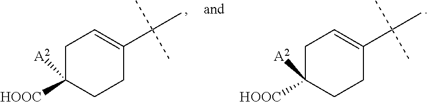

In accordance with another embodiment of the present invention, there is provided a compound of Formula I above, wherein

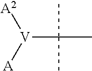

has the structure selected from the group consisting of the following:

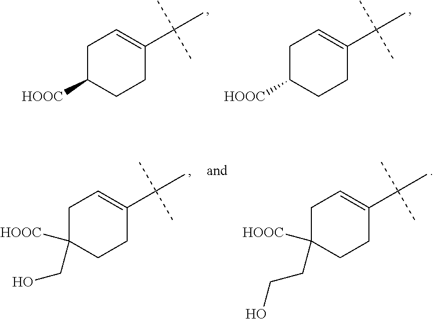

In accordance with another embodiment of the present invention, there is provided a compound having the structure of Formula I:

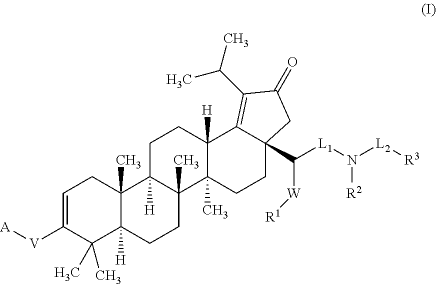

or a pharmaceutically acceptable salt thereof, wherein:

L₁ and L₂ are both (—CH₂—);

W is O;

R¹ is —H;

R² is selected from the group consisting of —(CH₂)₁NR⁷R⁸ and —C(O)R⁵;

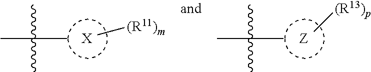

R³ is selected from the group consisting of

wherein:

X is phenyl,

Z is selected from the group consisting of cyclopropyl and cyclobutyl;

R⁵ is selected from the group consisting of —(CH₂)₁NR⁷R⁸, and —(CH₂)ᵣOR⁷;

R⁷ and R⁸ are independently selected from the group consisting of —H, methyl, wherein R⁷ and R⁸ can be taken together with the nitrogen to which they are joined to form a pyrrolidine ring or 2-pyrrolidone ring;

R¹¹ and R¹³ are independently selected from the group consisting of chloro, bromo, and fluoro;

m is 0, 1, or 2; and

p is 0, 1, or 2;

r is 1, 2, or 3.

In accordance with another embodiment of the present invention, there is provided a compound of Formula I above, wherein V is selected from a group consisting of —(C₄-C₈)cycloalkenyl, —(C₄-C₉)spirocycloalkyl, and —(C₄-C₉)spirocycloalkenyl.

In accordance with another embodiment of the present invention, there is provided a compound of Formula I above, wherein A is in the para position.

In accordance with another embodiment of the present invention, there is provided a compound of Formula I above, wherein A² is at least one member selected from the group the group consisting of —H, -halo, -hydroxyl, —(C₁-C₃)alkyl, —(C₁-C₃)alkoxy.

In accordance with another embodiment of the present invention, there is provided a compound of Formula I above, wherein A² is at least one member selected from the group group consisting of —H, —Cl, —F, and —Br.

In accordance with another embodiment of the present invention, there is provided a compound of Formula I above, wherein A² is at least one member selected from the group consisting of —F and —H.

In accordance with another embodiment of the present invention, there is provided a compound of Formula I above, wherein A² is —H.

In accordance with another embodiment of the present invention, there is provided a compound of Formula I above, wherein A is COOR¹⁷.

In accordance with another embodiment of the present invention, there is provided a compound of Formula I above, wherein A is —COOH.

In accordance with another embodiment of the present invention, there is provided a compound of Formula I above, wherein

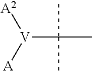

has the structure selected from the group consisting of the following:

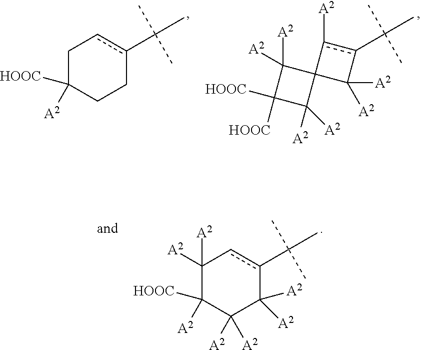

In accordance with another embodiment of the present invention, there is provided a compound of Formula I above, wherein

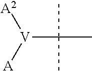

has the structure selected from the group consisting of the following:

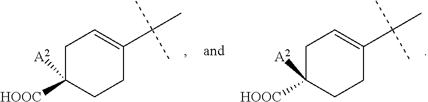

In accordance with another embodiment of the present invention, there is provided a compound of Formula I above, wherein

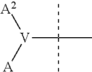

has the structure selected from the group consisting of the following:

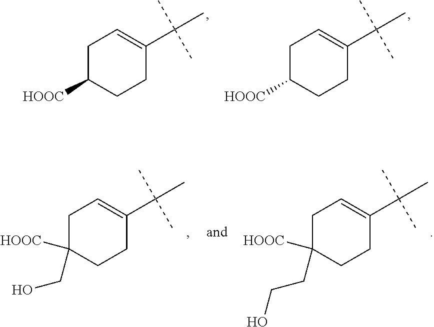

In accordance with another embodiment of the present invention, there is provided a compound of Formula I:

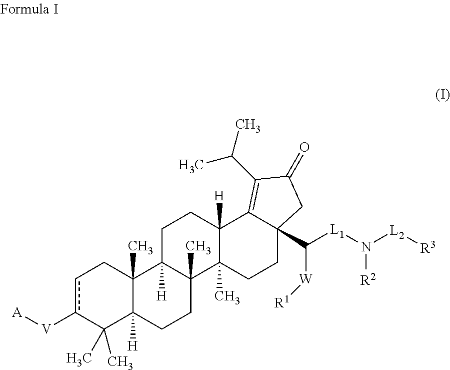

or a pharmaceutically acceptable salt thereof, wherein:

L₁ and L₂ are independently selected from a bond or [C(R⁶R^{6′})]_{q};

W is selected from a single bond or O;

R¹ is selected from the group consisting of —H, (C₁-C₁₂)alkyl, —C(O)R⁵, —CH₂—O—(C₁-C₆)alkyl, and 2-tetrahydro-2H-pyran;

R² is selected from the group consisting of —H, (C₁-C₁₂)alkyl, —(C₁-C₆)alkyl-OR⁴, —(C₁-C₆)alkyl-O—(C₁-C₆)alkyl, —C(O)R⁵, —(CH₂)₁NR⁷R⁸, and —(CH₂)₁N⁺(R⁴)₃, wherein when W is O, R¹ and R² can optionally be taken together with the O and N to which they are respectively joined to form a 4 to 8 membered heterocyclyl ring, wherein the heterocyclyl ring may be optionally substituted by one to two R¹¹ groups;

R³ is selected from the group consisting of hydrogen, (C₁-C₁₂)alkyl, —NR¹R², —OR⁵,

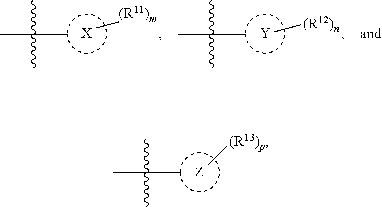

wherein:
X is a monocyclic or bicyclic (C₅-C₁₄)aryl,
Y is selected from a monocyclic or bicyclic (C₂-C₉)heterocyclyl or monocylic or bicyclic (C₂-C₉)heteroaryl, each having one to three heteroatoms selected from S, N or O, and
Z is a monocyclic or bicyclic (C₃-C₈)cycloalkyl;

R² and R³ can optionally be taken together with the nitrogen and L₂ to which they are respectively joined to form a 4 to 8 membered heterocyclyl ring, wherein the heterocyclyl ring may be optionally substituted by one to two R¹¹ groups;

R⁴ is selected from the group consisting of —H and (C₁-C₆)alkyl;

R⁵ is selected from the group consisting of —H, (C₁-C₆)alkyl, —R³, —(CH₂)₁NR⁷R⁸, and —(CH₂)ᵣOR⁷;

R⁶ and R^{6′} are independently selected from the group consisting of —H, (C₁-C₆)alkyl, (C₃-C₈)cycloalkyl, (C₁-C₆)alkoxy, haloalkyl, —Y, —(CH₂)₁NR⁷R⁸, —C(O)OH, and —C(O)NH₂, wherein the R⁶ and R^{6′} groups can optionally be taken together with the carbon to which they are joined to form a 3 to 8 membered cycloalkyl ring, and wherein the cycloalkyl ring may be optionally substituted by one to three R¹¹ groups;

R⁷ and R⁸ are independently selected from the group consisting of —H, (C₁-C₆)alkyl, (C₃-C₈)cycloalkyl, -Q-aryl-(R⁴)ₙ, —NR¹⁴R¹⁵, and —C(O)CH₃, wherein R⁷ and R⁸ can optionally be taken together with the nitrogen to which they are joined to form a 4 to 8 membered heterocyclyl or heteroaryl ring containing one to three heteroatoms selected from —NR⁵—, —O—, —S—, —S(O)—, or —SO₂—, wherein the heterocyclyl or heteroaryl ring may be optionally substituted by one to three R¹¹ groups;

R⁹ is halo;

R¹⁰ is —N(R¹⁶)₂;

R¹¹, R¹², and R¹³ are independently selected from the group consisting of oxo, hydroxyl, halo, (C₁-C₆)alkoxy, —R⁶(R⁹)_{q}, —OR⁶(R⁹)_{q}, nitro, —SO₂R⁶, (C₁-C₆)alkyl, —C(O)R¹⁰, —R⁴YR⁶, —CO(O)R⁴, and —CO(O)R⁵, wherein any two R¹¹, R¹² or R¹³ groups can optionally join to form a 3 to 8 membered cycloalkyl, aryl, heterocyclyl or heteroaryl ring, wherein the heterocyclyl or heteroaryl ring may contain one to three heteroatoms selected from —NR⁵—, —O—, —S—, —S(O)—, or —SO₂—, and wherein the cycloalkyl, aryl, heterocyclyl or heteroaryl ring may be optionally substituted by one to three R¹⁶ groups;

R¹⁴ and R¹⁵ are independently selected from the group consisting of —H, (C₁-C₆)alkyl, (C₃-C₈)cycloalkyl, (C₁-C₆)alkoxy, —[C(R⁶)₂]ᵣ—, —O[C(R⁶)₂]ᵣ—, oxo, hydroxyl, halo, —C(O)R⁷, —R¹⁰, and —CO(O)R², wherein R¹⁴ and R¹⁵ can optionally be taken together with the carbon to which they are joined to form a 3 to 8 membered cycloalkyl ring or 4 to 8 membered heterocyclyl ring containing one to three heteroatoms selected from —NR⁵—, —O—, -5-, —S(O)—, or —SO₂—, wherein the cycloalkyl ring or heterocyclyl ring may be optionally substituted by one to three R¹⁶ groups;

R¹⁶ is selected from the group consisting of —H, halo, oxo, hydroxyl, (C₁-C₆)alkyl, (C₁-C₆)alkoxy, (C₃-C₈)cycloalkyl, —R⁶(R⁹)_{q}, —OR⁶(R⁹)_{q}, —N(R⁴)₂, —(CH₂)ᵣ-heterocycle, —C(O)OH, —C(O)NH₂, —R⁵(R⁹)_{q}, —OR⁵(R⁹)_{q}, nitro, —SO₂R⁶, —C(O)R¹⁰, and —CO(O)R⁴;

V is selected from the group consisting of —(C₄-C₈)cycloalkyl, —(C₄-C₈)cycloalkenyl, —(C₄-C₉)spirocycloalkyl, —(C₄-C₈)spirocycloalkenyl, —(C₄-C₈)oxacycloalkyl, —(C₄-C₈)oxacycloalkenyl, —(C₄-C₈)dioxacycloalkyl, —(C₄-C₈)dioxacycloalkenyl, —C₆ cyclodialkenyl, —C₆ oxacyclodialkenyl, —(C₆-C₉)oxaspirocycloalkyl, —(C₆-C₉)oxaspirocycloalkenyl ring,

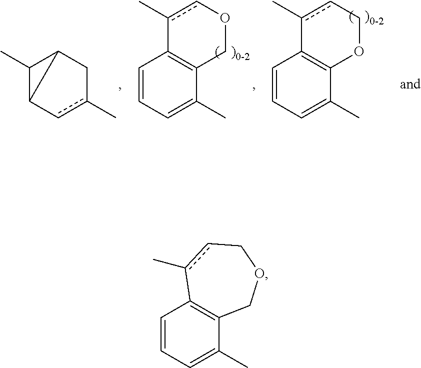

wherein:

V may be substituted with A², wherein:

A² is at least one member selected from the group consisting of —H, -halo, -hydroxyl, —(C₁-C₆)alkyl, —(C₁-C₆)alkoxy, —(C₁-C₆)alkyl-Q, -alkylsubstituted (C₁-C₆)alkyl-Q, —CN, —CF₂Q-NR¹⁷R¹⁷, —COOR¹⁷, and —CONR¹⁷R¹⁷, wherein:

Q is selected from the group consisting of aryl, heteroaryl, substituted heteroaryl, —OR¹⁷, —COOR¹⁸, —NR¹⁷R¹⁷, —SO₂R¹⁰, —CONHSO₂R¹⁸, and —CONHSO₂NR¹⁷R¹⁷;

R¹⁷ is selected from the group consisting of —H, —(C₁-C₆)alkyl, -alkylsubstituted (C₁-C₆)alkyl, and -arylsubstituted (C₁-C₆)alkyl;

R¹⁸ is selected from the group consisting of —(C₁-C₆)alkyl and -alkylsubstituted (C₁-C₆)alkyl;

R¹⁹ is selected from the group consisting of —(C₁-C₆)alkyl, —(C₁-C₆)substituted alkyl, —(C₃-C₆)cycloalkyl, —CF₃, aryl, and heteroaryl;

A is selected from the group consisting of —COOR¹⁷, —C(O)NR¹⁷SO₂R¹⁸, —C(O)NHSO₂NR¹⁷R¹⁷, —NR¹⁷SO₂R¹⁷, —SO₂NR¹⁷R¹⁷, —(C₃-C₆)cycloalkyl-COOR¹⁷, —(C₂-C₆)alkenyl-COOR¹⁷, —(C₂-C₆)alkynyl-COOR¹⁷, —(C₁-C₆)alkyl-COOR¹⁷, -alkylsubstituted (C₁-C₆)alkyl, —CF₂—COOR¹⁷, —NHC(O)(CH₂)ₙ₁—COOR¹⁷, —SO₂NR¹⁷C(O)R¹⁷, tetrazole, and —C(O)NHOH,

m and n in each instance are independently 0, 1, 2, 3, or 4;

p is independently 0, 1, 2, 3, or 4;

r and q in each instance are independently 0, 1, 2, 3, or 4; and

n¹ is independently 1, 2, 3, 4, 5, or 6.

In accordance with another embodiment of the present invention, there is provided a compound having the structure of Formula I above, wherein L₁ and L₂ are both) [C(R⁶R^{6′}]_{q}.

In accordance with another embodiment of the present invention, there is provided a compound having the structure of Formula I above, wherein L₁ and L₂ are both —CH₂—.

In accordance with another embodiment of the present invention, there is provided a compound having the structure of Formula I above, wherein q is independently 1, 2, or 3.

In accordance with another embodiment of the present invention, there is provided a compound having the structure of Formula I above, wherein q is 1.

In accordance with another embodiment of the present invention, there is provided a compound having the structure of Formula I above, wherein W is O.

In accordance with another embodiment of the present invention, there is provided a compound having the structure of Formula I above, wherein W is a bond.

In accordance with another embodiment of the present invention, there is provided a compound having the structure of Formula I above, wherein when W is a bond, then R¹ is —H.

In accordance with another embodiment of the present invention, there is provided a compound having the structure of Formula I above, wherein when W is O, then R¹ is —H.

In accordance with another embodiment of the present invention, there is provided a compound having the structure of Formula I above, wherein R¹ is —H.

In accordance with another embodiment of the present invention, there is provided a compound having the structure of Formula I above, wherein R² is selected from the group consisting of —H, —(CH₂)₁NR⁷R⁸, and —C(O)R⁵.

In accordance with another embodiment of the present invention, there is provided a compound having the structure of Formula I above, wherein R² is (dimethylamino)ethyl.

In accordance with another embodiment of the present invention, there is provided a compound having the structure of Formula I above, wherein R² is

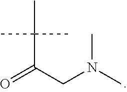

In accordance with another embodiment of the present invention, there is provided a compound having the structure of Formula I above, wherein R² is

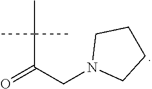

In accordance with another embodiment of the present invention, there is provided a compound having the structure of Formula I above, wherein R² is

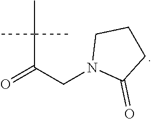

In accordance with another embodiment of the present invention, there is provided a compound having the structure of Formula I above, wherein R² is

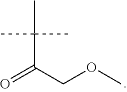

In accordance with another embodiment of the present invention, there is provided a compound having the structure of Formula I above, wherein R² is H.

In accordance with another embodiment of the present invention, there is provided a compound having the structure of Formula I above, wherein r is independently 0, 1, 2, or 3.

In accordance with another embodiment of the present invention, there is provided a compound having the structure of Formula I above, wherein r is 2.

In accordance with another embodiment of the present invention, there is provided a compound having the structure of Formula I above, wherein r is 1.

In accordance with another embodiment of the present invention, there is provided a compound having the structure of Formula I above, wherein R³ is

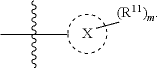

In accordance with another embodiment of the present invention, there is provided a compound having the structure of Formula I above, wherein X is a monocyclic (C₅-C₁₄)aryl.

In accordance with another embodiment of the present invention, there is provided a compound having the structure of Formula I above, wherein X is phenyl.

In accordance with another embodiment of the present invention, there is provided a compound having the structure of Formula I above, wherein R³ is

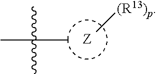

In accordance with another embodiment of the present invention, there is provided a compound having the structure of Formula I above, wherein Z is selected from the group cyclopropyl and cyclobutyl.

In accordance with another embodiment of the present invention, there is provided a compound having the structure of Formula I above, wherein Z is cyclopropyl.

In accordance with another embodiment of the present invention, there is provided a compound having the structure of Formula I above, wherein Z is cyclobutyl.

In accordance with another embodiment of the present invention, there is provided a compound having the structure of Formula I above, wherein m is 0 or 1.

In accordance with another embodiment of the present invention, there is provided a compound having the structure of Formula I above, wherein m is 0.

In accordance with another embodiment of the present invention, there is provided a compound having the structure of Formula I above, wherein m is 1.

In accordance with another embodiment of the present invention, there is provided a compound having the structure of Formula I above, wherein n is 1.

In accordance with another embodiment of the present invention, there is provided a compound having the structure of Formula I above, wherein p is 0 or 1.

In accordance with another embodiment of the present invention, there is provided a compound having the structure of Formula I above, wherein p is 0.

In accordance with another embodiment of the present invention, there is provided a compound having the structure of Formula I above, wherein R⁵ is selected from of —(CH₂)₁NR⁷R⁸ and —(CH₂)ᵣOR⁷.

In accordance with another embodiment of the present invention, there is provided a compound having the structure of Formula I above, wherein R⁵ is selected from the group consisting of

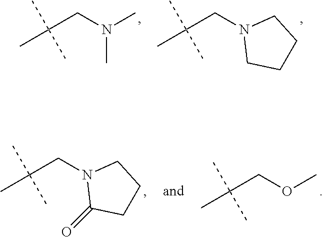

In accordance with another embodiment of the present invention, there is provided a compound having the structure of Formula I above, wherein R⁵ is

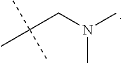

In accordance with another embodiment of the present invention, there is provided a compound having the structure of Formula I above, wherein R⁵ is

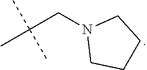

In accordance with another embodiment of the present invention, there is provided a compound having the structure of Formula I above, wherein R⁵ is

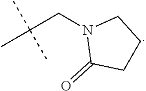

In accordance with another embodiment of the present invention, there is provided a compound having the structure of Formula I above, wherein R⁵ is

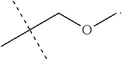

In accordance with another embodiment of the present invention, there is provided a compound having the structure of Formula I above, wherein R⁶ and R^{6′} are both —H

In accordance with another embodiment of the present invention, there is provided a compound having the structure of Formula I above, wherein R⁷ and R⁸ are both (C₁-C₆)alkyl.

In accordance with another embodiment of the present invention, there is provided a compound having the structure of Formula I above, wherein R⁷ and R⁸ are taken together with the nitrogen to which they are joined to form a group a heterocycle or heteroaryl ring.

In accordance with another embodiment of the present invention, there is provided a compound having the structure of Formula I above, wherein R⁷ and R⁸ are taken together with the nitrogen to which they are joined to form a group a heterocycle.

In accordance with another embodiment of the present invention, there is provided a compound having the structure of Formula I above, wherein R⁷ and R⁸ are taken together with the nitrogen to which they are joined to form a group selected from the following

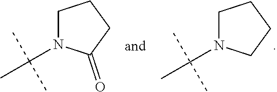

In accordance with another embodiment of the present invention, there is provided a compound having the structure of Formula I above, wherein R⁷ and R⁸ are taken together with the nitrogen to which they are joined to form

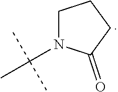

In accordance with another embodiment of the present invention, there is provided a compound having the structure of Formula I above, wherein R⁷ and R⁸ are taken together with the nitrogen to which they are joined to form

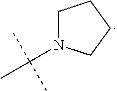

In accordance with another embodiment of the present invention, there is provided a compound having the structure of Formula I above, wherein R⁷ is methyl.

In accordance with another embodiment of the present invention, there is provided a compound having the structure of Formula I above, wherein R⁸ is methyl.

In accordance with another embodiment of the present invention, there is provided a compound having the structure of Formula I above, wherein R⁷ and R⁸ are both methyl.

In accordance with another embodiment of the present invention, there is provided a compound having the structure of Formula I above, wherein R¹¹ is halo.

In accordance with another embodiment of the present invention, there is provided a compound having the structure of Formula I above, wherein R¹¹ is selected from chloro, bromo, or fluoro.

In accordance with another embodiment of the present invention, there is provided a compound having the structure of Formula I above, wherein R¹¹ is chloro.

In accordance with another embodiment of the present invention, there is provided a compound having the structure of Formula I above, wherein R¹¹ is absent.

In accordance with another embodiment of the present invention, there is provided a compound having the structure of Formula I above, wherein R¹³ is selected from chloro, bromo, or fluoro.

In accordance with another embodiment of the present invention, there is provided a compound having the structure of Formula I above, wherein R¹³ is absent.

In accordance with another embodiment of the present invention, there is provided a compound having the structure of Formula I above, wherein V is selected from a group —(C₄-C₈)cycloalkenyl, —(C₄-C₉)spirocycloalkyl, and —(C₄-C₉)spirocycloalkenyl.

In accordance with another embodiment of the present invention, there is provided a compound having the structure of Formula I above, wherein A is in the para position.

In accordance with another embodiment of the present invention, there is provided a compound having the structure of Formula I above, wherein A is selected from the group consisting of —COOR¹⁷, —C(O)NR¹⁷SO₂R¹⁸, —C(O)NHSO₂NR¹⁷R¹⁷, —NR¹⁷SO₂R¹⁷, —SO₂NR¹⁷R¹⁷, —(C₃-C₆)cycloalkyl-COOR¹⁷, —(C₂-C₆)alkenyl-COOR¹⁷, —(C₂-C₆)alkynyl-COOR¹⁷, —(C₁-C₆)alkyl-COOR¹⁷, -alkylsubstituted (C₁-C₆)alkyl, —CF₂—COOR¹⁷, —NHC(O)(CH₂)ₙ₁—COOR¹⁷, —SO₂NR¹⁷C(O)R¹⁷, tetrazole, and —C(O)NHOH, wherein n¹=1-6.

In accordance with another embodiment of the present invention, there is provided a compound having the structure of Formula I above, wherein A is selected from the group consisting of —COOR¹⁷

In accordance with another embodiment of the present invention, there is provided a compound having the structure of Formula I above, wherein A is COOH.

In accordance with another embodiment of the present invention, there is provided a compound having the structure of Formula I above, wherein R¹⁷ is selected from the group consisting of —H, —(C₁-C₆)alkyl, -alkylsubstituted (C₁-C₆)alkyl, and -arylsubstituted (C₁-C₆)alkyl.

In accordance with another embodiment of the present invention, there is provided a compound having the structure of Formula I above, wherein R¹⁷ is —H.

In accordance with another embodiment of the present invention, there is provided a compound having the structure of Formula I above, wherein R¹⁸ is selected from the group consisting of —(C₁-C₆)alkyl and -alkylsubstituted (C₁-C₆)alkyl.

In accordance with another embodiment of the present invention, there is provided a compound having the structure of Formula I above, wherein A² is selected from the group consisting of —H, -halo, -hydroxyl, —(C₁-C₃)alkyl, and —(C₁-C₃)alkoxy.

In accordance with another embodiment of the present invention, there is provided a compound having the structure of Formula I above, wherein A² is selected from the group consisting of —H, —Cl, —FI, —Br, and —(C₁-C₃)alkoxy.

In accordance with another embodiment of the present invention, there is provided a compound having the structure of Formula I above, wherein A² is selected from the group consisting of —H, —FI, —CH₂OH, and —CH₂CH₂OH.

In accordance with another embodiment of the present invention, there is provided a compound having the structure of Formula I above, wherein A² is selected from the group consisting of —H and -Fl.

In accordance with another embodiment of the present invention, there is provided a compound having the structure of Formula I above, wherein A² is selected from the group consisting of —H.

In accordance with another embodiment of the present invention, there is provided a compound having the structure of Formula I above, wherein

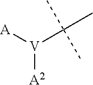

is selected from the group consisting of the following structures:

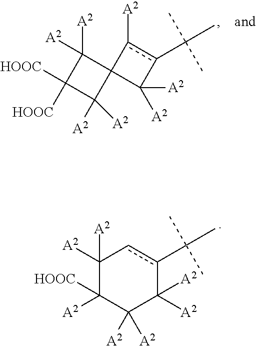

In accordance with another embodiment of the present invention, there is provided a compound having the structure of Formula I above, wherein

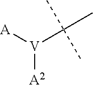

is selected form the group consisting of the following structures:

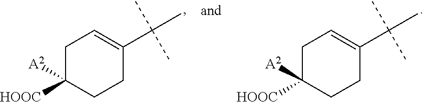

In accordance with another embodiment of the present invention, there is provided a compound having the structure of Formula I above, wherein

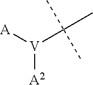

is selected form the group consisting of the following structures:

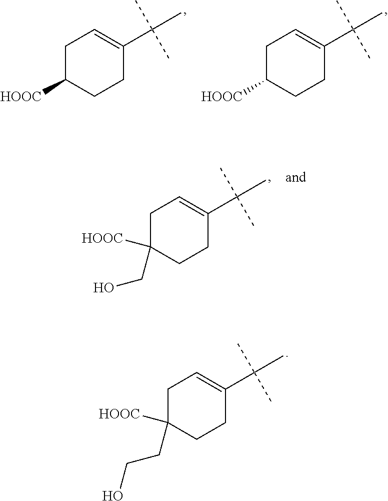

In a further embodiment of the present invention, there is provided a pharmaceutical composition comprising a compound of Formula I, or a pharmaceutically acceptable salt thereof; and a pharmaceutically acceptable excipient.

In a further embodiment of the present invention, there is provided a method of treating HIV comprising administering to a patient suffering there from an effective amount of a compound of Formula I, or a pharmaceutically acceptable salt thereof.

In a further embodiment of the present invention, there is provided a pharmaceutical composition comprising a compound of Formula I, or a pharmaceutically acceptable salt thereof, and a pharmaceutically acceptable excipient.

In a further embodiment of the present invention, there is provided a pharmaceutical composition comprising a compound of Formula I, or a pharmaceutically acceptable salt thereof, and a pharmaceutically acceptable excipient, wherein the compound is present in an amorphous form.

In a further embodiment of the present invention, there is provided a pharmaceutical composition comprising a compound of Formula I, or a pharmaceutically acceptable salt thereof, and a pharmaceutically acceptable excipient, wherein the composition is in a tablet form.

In a further embodiment of the present invention, there is provided a pharmaceutical composition comprising a compound of Formula I, or a pharmaceutically acceptable salt thereof, and a pharmaceutically acceptable excipient, wherein the compound is present as a spray dried dispersion.

In a further embodiment of the present invention, there is provided a method of treating an HIV infection in a subject comprising administering to the subject a compound of Formula I, or a pharmaceutically acceptable salt thereof. In certain embodiments, the subject is a mammal, and in other embodiments, the subject is a human.

In a further embodiment of the present invention, there is provided a method of treating an HIV infection in a subject comprising administering to the subject a pharmaceutical composition comprising a compound of Formula I, or a pharmaceutically acceptable salt thereof, and a pharmaceutically acceptable excipient.

In a further embodiment of the present invention, there is provided a method of preventing an HIV infection in a subject at risk for developing an HIV infection, comprising administering to the subject a compound of Formula I, or a pharmaceutically acceptable salt thereof.

In a further embodiment of the present invention, there is provided a method of preventing an HIV infection in a subject at risk for developing an HIV infection, comprising administering to the subject a pharmaceutical composition comprising a compound of Formula I, or a pharmaceutically acceptable salt thereof, and a pharmaceutically acceptable excipient.

In still other embodiments, the present invention also provides the use of a compound or salt as defined in any of Formula I in the manufacture of a medicament for use in the treatment of an HIV infection in a human.

Furthermore, the compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and I isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

In another embodiment of the invention, there is provided a compound of Formula I, wherein the compound or salt of the compound is used in the manufacture of a medicament for use in the treatment of a viral infection in a human.

In another embodiment of the invention, there is provided a pharmaceutical composition comprising a pharmaceutically acceptable diluent and a therapeutically effective amount of a compound as defined in Formula I.

In one embodiment, the pharmaceutical formulation containing a compound of Formula I or a salt thereof is a formulation adapted for parenteral administration. In another embodiment, the formulation is a long-acting parenteral formulation. In a further embodiment, the formulation is a nano-particle formulation.

The compounds of the present invention and their salts, solvates, or other pharmaceutically acceptable derivatives thereof, may be employed alone or in combination with other therapeutic agents. Therefore, in other embodiments, the methods of treating and/or preventing an HIV infection in a subject may in addition to administration of a compound of Formula I further comprise administration of one or more additional pharmaceutical agents active against HIV.

In such embodiments, the one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, etravirine, rilpivirine, enfuvirtide, saquinavir, ritonavir, indinavir, nelfinavir, amprenavir, fosamprenavir, brecanavir, darunavir, atazanavir, tipranavir, palinavir, lasinavir, enfuvirtide, T-1249, PRO-542, PRO-140, BMS-806, fostemsavir, and temsavir, 5-Helix, raltegravir, elvitegravir, dolutegravir, cabotegravir, vicriviroc, TAK779, maraviroc, TAK449, didanosine, tenofovir disoproxil fumarate, lopinavir, dexelvucitabine, festinavir, racivir, doravirine, rilpivirine, ibalizumab, cenicriviroc, INCB-9471, monomeric DAPTA, AMD-070, ibalizumab, and darunavir.

As such, the compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of Formula I or salts thereof and the other pharmaceutically active agent(s) and the relative timings of administration will be selected in order to achieve the desired combined therapeutic effect.

In addition, the compounds of the present invention may be used in combination with one or more other agents useful in the prevention or treatment of HIV.

Examples of such agents include:

Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, tenofovir disoproxil fumarate, dexelvucitabine, festinavir, racivir, tenofovir alafenamide, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, doravirine, rilpivirine, GSK2248761, etravirine, and similar agents;

Protease inhibitors such as saquinavir, ritonavir, indinavir, nelfinavir, amprenavir, fosamprenavir, brecanavir, darunavir, atazanavir, tipranavir, palinavir, lasinavir, and similar agents;

Entry, attachment and fusion inhibitors such as enfuvirtide (T-20), T-1249, PRO-542, PRO-140, ibalizumab, cenicriviroc, INCB-9471, monomeric DAPTA, AMD-070, ibalizumab, BMS-806, fostemsavir, temsavir, and 5-Helix and similar agents;

Integrase strand transfer inhibitors such as raltegravir, elvitegravir, dolutegravir, cabotegravir, GS-9883 and similar agents;

Maturation inhibitors such as PA-344, PA-457, BMS-955176, as well as those disclosed in PCT Patent Application No. WO2011/100308, PCT Patent Application No. PCT/US2012/024288, Chinese PCT Application No. PCT/CN2011/001302, Chinese PCT Application No. PCT/CN2011/001303, Chinese PCT Application No. PCT/CN2011/002105, PCT/CN2011/002159, WO2013/090664, WO2013/123019, WO 2013/043778, WO 2014/123889, WO 2011/153315, WO 2011/153319, WO 2012/106188, WO 2012/106190, WO 2013/169578, WO 2014/13081, and similar agents;

CXCR4 and/or CCR5 inhibitors such as vicriviroc, TAK779, maraviroc, TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

Neutralizing antibodies such as VRC01, VRC07 10e8, pro140, PGT121, PGT128, PGT145, PG9, 3BNC117, N6, and ibalizumab, and similar agents.

In addition, the compounds of the present invention maybe used in combination with one or more of the following agents useful in the prevention or treatment of HIV including but not limited to: valproic acid, vorinostat, tucersol, SB-728-T, astodrimer, carbopol 974P, carrageenan, dapivirine, PRO-2000, and tenofovir.

Further examples wherein the compounds of the present invention may be used in combination with one or more agents useful in the prevention or treatment of HIV are found in Table 1.

TABLE 1

| | | | |
|---|---|---|---|
| FDA | Brand | | |
| Approval | Name | Generic Name | Manufacturer |

Nucleoside Reverse Transcriptase Inhibitors (NRTIs)

| | | | |
|---|---|---|---|
| 1987 | Retrovir | zidovudine, | GlaxoSmithKline |
| | | azidothymidine, | |
| | | AZT, ZDV | |
| 1991 | Videx | didanosine, | Bristol-Myers |
| | | dideoxyinosine, ddI | Squibb |
| 1992 | Hivid | zalcitabine, | Roche |
| | | dideoxycytidine, | Pharmaceuticals |
| | | ddC | |
| 1994 | Zerit | stavudine, d4T | Bristol-Myers |
| | | | Squibb |
| 1995 | Epivir | lamivudine, 3TC | GlaxoSmithKline |
| 1998 | Ziagen | abacavir sulfate, | GlaxoSmithKline |
| | | ABC | |
| 2000 | Videx EC | enteric coated | Bristol-Myers |
| | | didanosine, ddI EC | Squibb |
| 2001 | Viread | tenofovir disoproxil | Gilead Sciences |
| | | fumarate, TDF | |
| 2003 | Emtriva | emtricitabine, FTC | Gilead Sciences |

Non-Nucleosides Reverse Transcriptase Inhibitors (NNRTIs)

| | | | |
|---|---|---|---|
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | etravirine | Tibotec |
| | | | Therapeutics |
| 2011 | Viramune | Extended-release | Boehringer |
| | XR | nevirapine, NVP | Ingelheim |
| 2011 | Edurant | rilpivirine | Janseen |
| | | hydrochloride, RPV | Therapeutics |

Protease Inhibitors (PIs)

| | | | |
|---|---|---|---|
| 1995 | Invirase | saquinavir | Roche |
| | | mesylate, SQV | Pharmaceuticals |
| 1996 | Norvir | ritonavir, RTV | Abbott Laboratories |
| 1996 | Crixivan | indinavir, IDV | Merck |
| 1997 | Viracept | nelfinavir mesylate, | Pfizer |
| | | NFV | |
| 1997 | Fortovase | saquinavir (no | Roche |
| | | longer marketed) | Pharmaceuticals |
| 1999 | Agenerase | amprenavir, APV | GlaxoSmithKline |
| 2000 | Kaletra | lopinavir + ritonavir, | Abbott Laboratories |
| | | LPV/RTV | |
| 2003 | Reyataz | atazanavir sulfate, | Bristol-Myers |
| | | ATV | Squibb |
| 2003 | Lexiva | fosamprenavir | GlaxoSmithKline |
| | | calcium, FOS-APV | |
| 2005 | Aptivus | tripranavir, TPV | Boehringer |
| | | | Ingelheim |
| 2006 | Prezista | darunavir | Tibotec |
| | | | Therapeutics |

Fusion Inhibitors

| | | | |
|---|---|---|---|
| 2003 | Fuzeon | Enfuvirtide, T-20 | Roche |
| | | | Pharmaceuticals & |
| | | | Trimeris |

Entry Inhibitors

| | | | |
|---|---|---|---|
| 2007 | Selzentry | maraviroc | Pfizer |

Integrase Inhibitors

| | | | |
|---|---|---|---|
| 2007 | Isentress | raltegravir | Merck |
| 2013 | Tivicay | Dolutegravir, DIG | ViiV Healthcare |
| 2014 | Vitekta | Elvitegravir, EVG | Gilead |

Combination HIV Medicines

| | | | |
|---|---|---|---|
| 1997 | Combivir | lamivudine + | GlaxoSmithKline |
| | | zidovudine | |
| 2000 | Trizivir | abacavir + | GlaxoSmithKline |
| | | lamivudine + | |
| | | zidovudine | |
| 2004 | Epzicom | abacavir + | GlaxoSmithKline |
| | | lamivudine | |
| 2004 | Truvada | emtricitabine + | Gilead Sciences |
| | | tenofovir disoproxil | |
| | | fumarate | |
| 2006 | Atripla | Efavirenz + | Bristol-Myers |
| | | emtricitabine + | Squibb and Gilead |
| | | tenofovir | Sciences |
| 2011 | Complera | Emtricitabine + | Gilead Sciences |
| | | Rilpivirine + | |
| | | tenofovir disoproxil | |
| | | fumarate | |
| 2012 | Stribild | Elvitegravir + | Gilead Sciences |
| | | cobicistat + | |
| | | emtricitabine + | |
| | | tenofovir disoproxil | |
| | | fumarate | |
| 2014 | Triumeq | abacavir + | ViiV Healthcare |
| | | dolutegravir + | |
| | | lamivudine | |
| 2015 | Evotaz | Atazanavir + | Bristol-Myers |
| | | cobicistat | Squibb |
| 2015 | Prezcobix | Darunavir + | Janssen |
| | | cobicistat | |

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be prior to, concurrent to, or subsequent to the administration of other agent(s).

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir and Cobicistat (formerly GS-9350).

Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott Park, Ill., as Norvir. Ritonavir is an HIV protease inhibitor indicated with other antiretroviral agents for the treatment of HIV infection. Ritonavir also inhibits P450 mediated drug metabolism as well as the P-gycoprotein (Pgp) cell transport system, thereby resulting in increased concentrations of active compound within the organism.

Cobicistat (formerly GS-9350) is thiazol-5-ylmethyl N-[1-benzyl-4-[[2-[[(2-isopropylthiazol-4-yl)methyl-methyl-carbamoyl]amino]-4-morpholino-butanoyl]amino]-5-phenyl-pentyl]carbamate and is available from Gilead Sciences of Foster City, Calif., as Tybost. Cobicistat is a potent inhibitor of cytochrom P450 3A enzymes, including the important CYP3A4 stubtype. It also inhibits intestinal transport proteins, thereby resulting in increased overall absorption of active compounds within the organism.

In one embodiment of the present invention, a compound of Formula I is used in combination with ritonavir. In one embodiment, the combination is an oral fixed dose combination. In another embodiment, the compound of Formula I is formulated as a long acting parenteral injection and ritonavir is formulated as an oral composition. In one embodiment, is a kit containing the compound of Formula I formulated as a long acting parenteral injection and ritonavir formulated as an oral composition. In another embodiment, the compound of Formula I is formulated as a long acting parenteral injection and ritonavir is formulated as an injectable composition. In one embodiment, is a kit containing the compound of Formula I formulated as a long acting parenteral injection and ritonavir formulated as an injectable composition.

In another embodiment of the present invention, a compound of Formula I is used in combination with cobicistat. In one embodiment, the combination is an oral fixed dose combination. In another embodiment, the compound of Formula I is formulated as a long acting parenteral injection and cobicistat is formulated as an oral composition. In one embodiment, there is provided a kit containing the compound of Formula I formulated as a long acting parenteral injection and cobicistat formulated as an oral composition. In another embodiment, the compound of Formula I is formulated as a long acting parenteral injection and cobicistat is formulated as an injectable composition. In one embodiment, is a kit containing the compound of Formula I is formulated as a long acting parenteral injection and cobicistat formulated as an injectable composition.

The above other therapeutic agents, when employed in combination with the chemical entities described herein, may be used, for example, in those amounts indicated in the Physicians' Desk Reference (PDR) or as otherwise determined by one of ordinary skill in the art.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula I.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula I, wherein said virus is an HIV virus. In some embodiments, the HIV virus is the HIV-1 virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula I, further comprising administration of a therapeutically effective amount of one or more agents active against an HIV virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula I, further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In further embodiments, the compound of the present invention, or a pharmaceutically acceptable salt thereof, is chosen from the compounds set forth in Table 2. Wherein a salt is indicated in Table 2, the present invention also encompasses the free base.

TABLE 2

| Example | Compound | | |
|---|---|---|---|
| No. | No. | Parent Structure | Chemical Name |
| 1 | 21 | 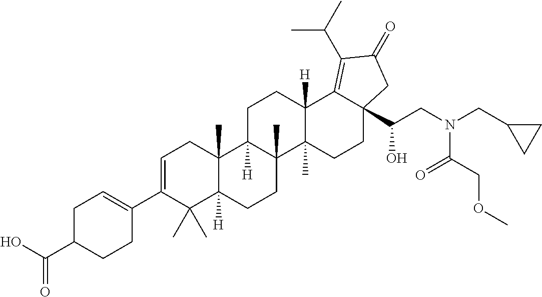 | 4- ((3aR,5aR,5bR,7aR,11aS, 11bR,13aS)-3a-((R)-2- (N-(cyclopropylmethyl)- 2-methoxyacetamido)-1- hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |
| 2 | 22 | 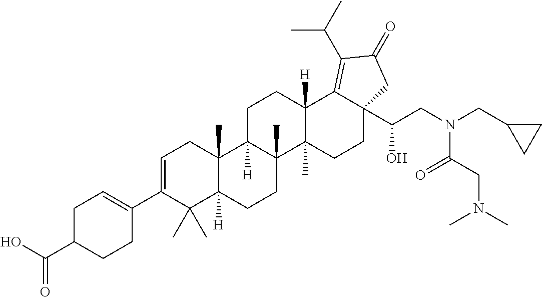 | 4- ((3aR,5aR,5bR,7aR,11aS, 11bR,13aS)-3a-((R)-2- (N-(cyclopropylmethyl)- 2- (dimethylamino)acetamido)- 1-hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |
| 3 | 23 | 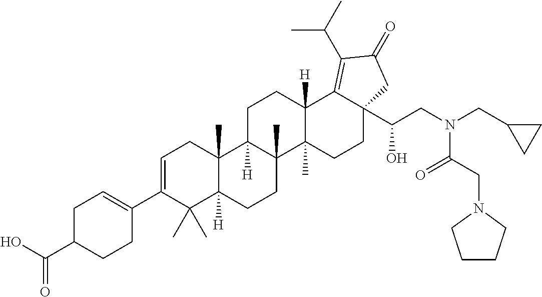 | 4- ((3aR,5aR,5bR,7aR,11aS, 11bR,13aS)-3a-((R)-2- (N-(cyclopropylmethyl)- 2-(pyrrolidin-1- yl)acetamido)-1- hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |
| 4 | 24 | 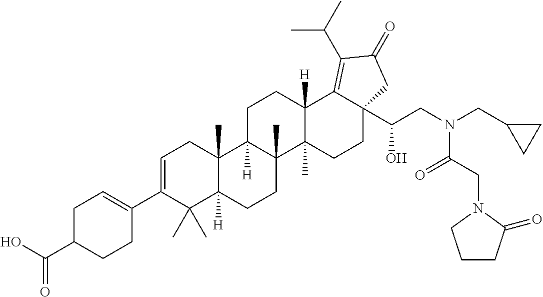 | 4- ((3aR,5aR,5bR,7aR,11aS, 11bR,13aS)-3a-((R)-2- (N-(cyclopropylmethyl)- 2-(2-oxopyrrolidin-1- yl)acetamido)-1- hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |
| 5 | 25 | 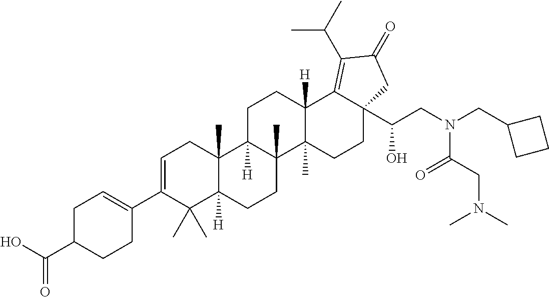 | 4- ((3aR,5aR,5bR,7aR,11aS, 11bR,13aS)-3a-((R)-2- (N-(cyclobutylmethyl)-2- (dimethylamino)acetamido)- 1-hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |
| 6 | 26 | 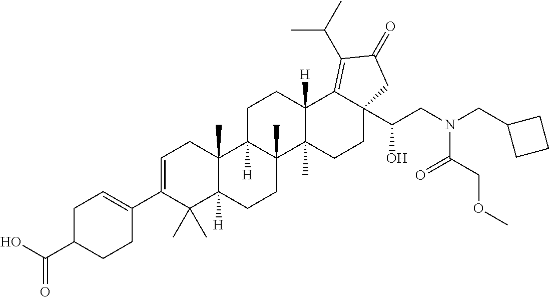 | 4- ((3aR,5aR,5bR,7aR,11aS, 11bR,13aS)-3a-((R)-2- (N-(cyclobutylmethyl)-2- methoxyacetamido)-1- hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |
| 7 | 27 | 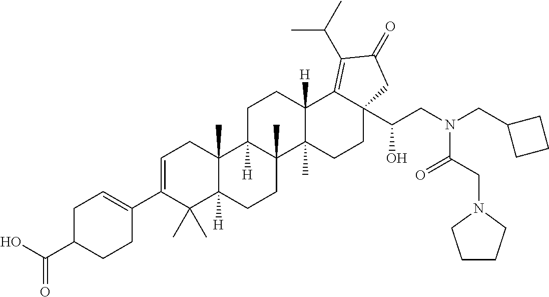 | 4- ((3aR,5aR,5bR,7aR,11aS, 11bR,13aS)-3a-((R)-2- (N-(cyclobutylmethyl)-2- (pyrrolidin-1- yl)acetamido)-1- hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |
| 8 | 28 | 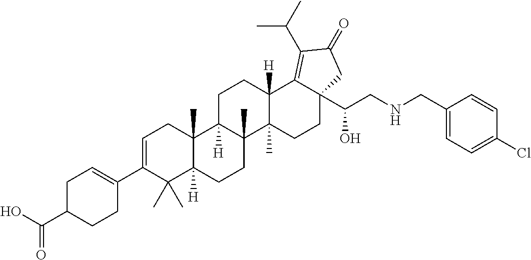 | 4- ((3aR,5aR,5bR,7aR,11aS, 11bR,13aS)-3a-((R)-2- ((4-chlorobenzyl)amino)- 1-hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |
| 9 | 29 | 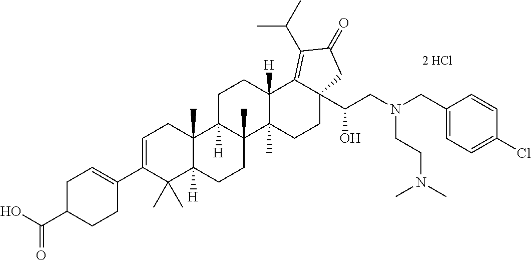 | 4- ((3aR,5aR,5bR,7aR,11aS, 11bR,13aS)-3a-((R)-2- ((4-chlorobenzyl)(2- (dimethylamino)ethyl) amino)-1-hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid dihydrochloride |
| 10 | 36 | 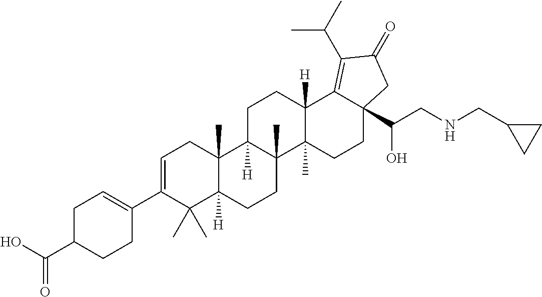 | 4- ((3aR,5aR,5bR,7aR,11aS, 11bR,13aS)-3a-((R)-2- ((cyclopropylmethyl) amino)-1-hydroxyethyl)-1- isopropyl-5a,5b,8,8,11a- pentamethyl-2-oxo- 3,3a,4,5,5a,5b,6,7,7a,8, 11,11a,11b,12,13,13a- hexadecahydro-2H- cyclopenta[a]chrysen-9- yl)cyclohex-3- enecarboxylic acid |

The compounds of Table 2 were synthesized according to the Synthetic Methods, General Schemes, and the Examples described below. Any chemical not directly described are readily prepared by one skilled in the art using available starting materials.

In certain embodiments, the compound(s) of the present invention, or a pharmaceutically acceptable salt thereof, is chosen from the compounds set forth in Table 2. Wherein a salt is indicated in Table 2, the present invention also encompasses the free base.

### Synthetic Methods

The methods of synthesis for the provided chemical entities employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein take place at atmospheric pressure, generally within a temperature range from −78° C. to 200° C. Further, except as employed in the Examples or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (or dichloromethane), diethyl ether, methanol, N-methylpyrrolidone (“NMP”), pyridine and the like.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### EXAMPLES

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.
aq.=aqueous
μL=microliters
μM=micromolar
NMR=nuclear magnetic resonance
boc=tert-butoxycarbonyl
br=broad
Cbz=benzyloxycarbonyl
d=doublet
δ=chemical shift
° C.=degrees celcius
DCE=1,2-dichloroethene
DCM=dichloromethane
dd=doublet of doublets
DIEA or DIPEA=N,N-diisopropylethylamine
DMEM=Dulbeco's Modified Eagle's Medium
DMF=N,N-dimethylformamide
DMP=Dess-Martin periodinane
DMSO=dimethylsulfoxide
EtOAc=ethyl acetate
FA=formic acid
g=gram
h or hr=hours
HBTU=2-(1H-benzotriazol-1-yl)-1,1,3,3-tetramethyluroniurn hexafluorophosphate
HCV=hepatitus C virus
HPLC=high performance liquid chromatography
Hz=hertz
IU=International Units
IC₅₀=inhibitory concentration at 50% inhibition
J=coupling constant (given in Hz unless otherwise indicated)
K-HMDS=potassium bis(trimethylsilyl)amide
m=multiplet
M=molar
M+H⁺=parent mass spectrum peak plus H⁺
mg=milligram
min=minutes
mL=milliliter
mM=millimolar
mmol=millimole
MS=mass spectrum
nm=nanomolar
PE=petroleum ether
ppm=parts per million
q.s.=sufficient amount
s=singlet
RT=room temperature
sat.=saturated
t=triplet
TBAF=tetra-n-butylammonium fluoride
TBSCl=tert-butyldimethylsilyl chloride
TEA=triethylamine
TFA=trifluoroacetic acid
THF=tetrahydrofuran

### Equipment Description

¹H NMR spectra were recorded on a Bruker Ascend 400 spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Waters ACQUITY UPLC with SQ Detectors using a Waters BEH C18, 2.1×50 mm, 1.7 μm using a gradient elution method.

Solvent A: 0.1% formic acid (FA) in water;

Solvent B: 0.1% FA in acetonitrile;

30% B for 0.5 min followed by 30-100% B over 2.5 min.

### Schemes and Experimental Procedures

The following schemes and procedures illustrate how compounds of the present invention can be prepared. The specific solvents and reaction conditions referred to are also illustrative and are not intended to be limiting. Compounds not described are either commercially available or are readily prepared by one skilled in the art using available starting materials. The Examples disclosed herein are for illustrative purposes only and are not intended to limit the scope of the invention. All examples exhibited LHIV IC₅₀ values between 1 μM and 1 nM using the assay disclosed herein.

For several of the examples the stereochemistry of the C28 secondary alcohol when present was not definitively confirmed as to its absolute configuration. Unless stated otherwise, the compounds exemplified in the present application were isolated as optically pure stereoisomers and initially assigned to a configuration as drawn. There is the possibility that some of these may be listed as the opposite stereochemistry at that single C28 position as shown. This in no way is meant to limit the scope of the invention or utility of the compounds of Formula I. Additional examples contained within were determined to have the shown configuration by spectroscopic methods well known to those skilled in the art including, but not limited to, 1D and 2D NMR methods, vibrational circular dichroism and X-ray crystallography. These examples and the methods to make both diastereomers should serve to clearly exemplify the pure stereoisomers of both R and S configuration at the C28 position are readily obtained, separated and characterized and any remaining undefined examples could be readily confirmed by similar methods well known to one skilled in the art.

Synthesis of Intermediate 5.

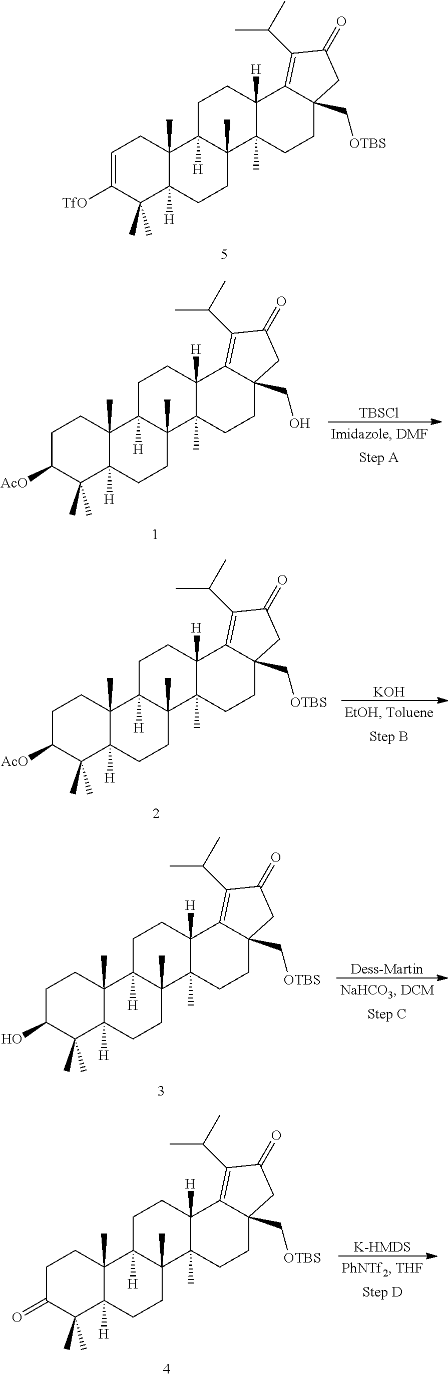

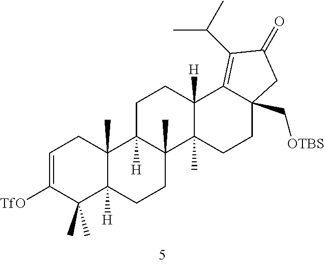

### Step A: Intermediate 2

(3aR,5aR,5bR,7aR, 9S,11aR,11bR,13aS)-3a-(((tert-Butyldimethylsilyl)oxy)methyl)-1-isopropyl-5a,5b,8,8,11a-pentamethyl-2-oxo-3,3a, 4,5,5a,5b,6,7,7a,8,9,10,11,11a,11b, 12,13,13a-octadecahydro-2H-cyclopenta[a]chrysen-9-yl acetate

To a solution of intermediate 1 (WO 2013/090664) (9 g, 18.05 mmol) in DMF (90 mL) was added imidazole (1.96 g, 28.9 mmol) and TBSCl (4.08 g, 27.1 mmol). After stirred at room temperature for 4 hr, the resulting mixture was diluted with H₂O and extracted with EtOAc. The organic layer was washed with brine, dried over Na₂SO₄, filtered and concentrated under reduced pressure to give intermediate 2 (11.9 g, quant. yield) which was used in the next step without further purification. ¹H NMR (400 MHz, CDCl₃) δ 4.49 (dd, J=10.9, 5.5 Hz, 1H), 3.62 (dd, J=39.0, 9.5 Hz, 2H), 3.15 (dt, J=13.9, 6.9 Hz, 1H), 2.74 (dd, J=11.9, 3.9 Hz, 1H), 2.43 (d, J=18.5 Hz, 1H), 2.05 (s, 3H), 1.32 (m, 49H), 0.01 (d, J=2.2 Hz, 6H).

### Step B: Intermediate 3

### (3aR,5aR,5bR,7aR,9S,11aR,11bR,13aS)-3a-(((tert-Butyldimethylsilyl)oxy)methyl)-9-hydroxy-1-isopropyl-5a,5b,8,8,11a-pentamethyl-3,3a,4,5,5a, 5b,6,7,7a,8,9,10,11,11a,11b,12,13,13a-octadecahydro-2H-cyclopenta[a]chrysen-2-one

A mixture of intermediate 2 (11.9 g, 19.4 mmol) and KOH (4.36 g, 77.7 mmol) in EtOH (120 mL) and toluene (120 mL) was stirred at room temperature overnight. The resulting mixture was neutralized with 1N HCl and concentrated reduced pressure to move the volatiles. The residue was partitioned between DCM and H₂O and the layers were separated. The organic layer was washed with brine, dried over Na₂SO₄, filtered and concentrated under reduced pressure to give intermediate 3 (10.7 g, 96% yield) which was directly used in the next step without further purification. ¹H NMR (400 MHz, CDCl3) δ 3.63 (dd, J=42.7, 9.5 Hz, 2H), 3.18 (m, 2H), 2.75 (dd, J=12.2, 3.9 Hz, 1H), 2.43 (d, J=18.5 Hz, 1H), 1.30 (m, 50H), 0.02 (d, J=2.2 Hz, 6H).

### Step C: Intermediate 4

### (3aR,5aR,5bR,7aR,11aR,11bR,13aS)-3a-(((tert-Butyldi methylsilyl)oxy)methyl)-1-isopropyl-5a, 5b,8,8,11a-pentamethyl-3a,4,5,5a, 5b,6,7,7a,8,10,11,11a,11b,12,13,13a-hexadecahydro-2H-cyclopenta[a]chrysene-2,9(3H)-dione

To a solution of intermediate 3 (10.7 g, 18.7 mmol) in DCM (120 mL) was added NaHCO₃ (15.7 g, 187 mmol) and DMP (15.9 g, 37.5 mmol). After stirred at room temperature for 4 hr, the resulting mixture was diluted with DCM and washed with sat. Na₂S₂O₃ solution. The layers were separated and the organic layer was washed with brine, dried over Na₂SO₄, filtered and concentrated under reduced pressure to give the crude product which was purified by silica gel chromatography (0-10% EtOAc/PE) to afford intermediate 4 (8.4 g, 79% yield) as a white solid. ¹H NMR (400 MHz, CDCl₃) δ 3.62 (dd, J=45.4, 9.5 Hz, 2H), 3.13 (m, 1H), 2.76 (dd, J=12.1, 3.8 Hz, 1H), 2.47 (m, 3H), 1.38 (m, 47H), 0.01 (d, J=1.9 Hz, 6H).

### Step D: Intermediate 5

### (3aR,5aR,5bR,7aR,11aR,11bR,13aS)-3a-(((tert-Butyldimethylsilyl)oxy)methyl)-1-isopropyl-5a,5b,8,8,11a-pentamethyl-2-oxo-3,3a, 4,5,5a,5b, 6,7,7a,8,11,11a,11b,12,13,13a-hexadecahydro-2H-cyclopenta[a]chrysen-9-yl trifluoromethanesulfonate

At −78° C., to a solution of intermediate 4 (8.4 g, 14.8 mmol) in anhydrous THF (105 mL) was added K-HMDS (22.2 mL, 1M in THF, 22.2 mmol). The reaction mixture was kept at −78° C. for 1 hr and a solution of PhNTf₂ (7.9 g, 22.2 mmol) in THF (63 mL) was added to the reaction. The resulting mixture was warmed up to room temperature and stirred for 2 hr before the completion of the reaction. The reaction was quenched with sat. NH₄Cl solution and extracted with EtOAc. The organic layer was washed with brine, dried over Na₂SO₄, filtered and concentrated under reduced pressure to give the crude product which was purified by silica gel chromatography (0-10% EtOAc/PE) to afford intermediate 5 (6.5 g, 63% yield) as a white solid. ¹H NMR (400 MHz, CDCl₃) δ 5.59 (dd, J=6.7, 1.8 Hz, 1H), 3.64 (dd, J=53.7, 9.5 Hz, 2H), 3.15 (dt, J=13.9, 7.0 Hz, 1H), 2.78 (dd, J=12.3, 3.6 Hz, 1H), 2.45 (d, J=18.5 Hz, 1H), 2.25 (dd, J=17.0, 6.8 Hz, 1H), 1.88 (m, 6H), 1.25 (m, 40H), 0.02 (d, J=1.1 Hz, 6H).

Synthesis of the boronate intermediates 10 and 11 was accomplished according to the following procedures.

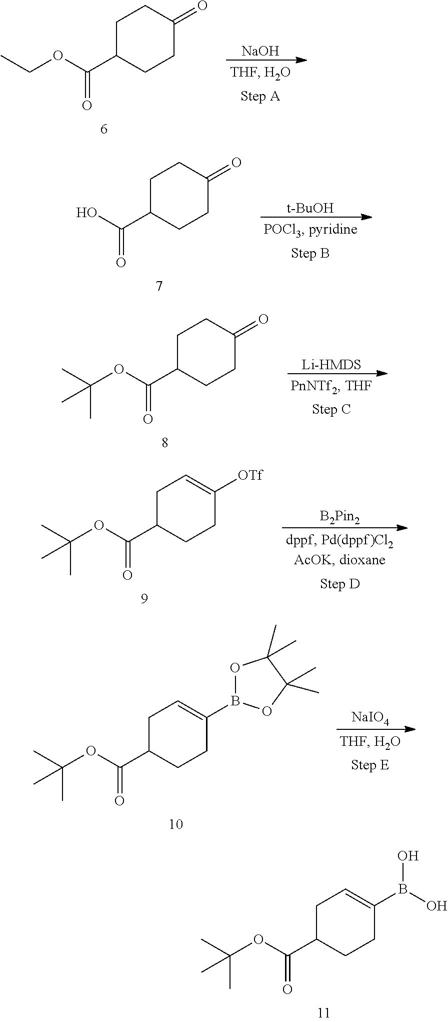

### Step A: Intermediate 7

### 4-oxocyclohexanecarboxylic acid

To a solution of ethyl 4-oxocyclohexane-1-carboxylate, intermediate 6 (20 g, 117 mmol) in a mixture of MeOH (120 mL) and THF (500 mL) was added an aqueous solution of NaOH (3N, 117 mL, 351 mmol) and the resulting mixture was heated at 60° C. for 3 hr. After cooled down to room temperature, the reaction mixture was concentrated under reduced pressure and the residue was acidified with 1N HCl to pH=1 and extracted with EtOAc. The organic layer was washed with brine, dried over Na₂SO₄, filtered and concentrated under reduced pressure to give intermediate 7 (13 g, 78% yield). ¹H NMR (400 MHz, CDCl₃) δ 11.23 (br, 1H), 2.82 (tt, J=9.5, 4.0 Hz, 1H), 2.51 (dt, J=14.7, 5.5 Hz, 2H), 2.38 (m, 2H), 2.26 (ddd, J=13.2, 8.7, 4.5 Hz, 2H), 2.06 (m, 2H). LC/MS: m/z calculated 142.2, found 143.3 (M+1)+.

### Step B: Intermediate 8

### tert-butyl 4-oxocyclohexanecarboxylate

To an ice-cold solution of intermediate 7 (5.0 g, 35 mmol) in pyridine (19 mL) and t-BuOH (27 mL) was added POCl₃ (4.7 mL, 50.6 mmol). The reaction mixture was warmed up to room temperature and stirred for 4 hr. The crude mixture was poured into ice water and extracted with EtOAc. The organic layer was washed with brine, dried over Na₂SO₄, filtered and concentrated under reduced pressure to give intermediate 8 (4.0 g, 58% yield) which was used in the next step without further purification. ¹H NMR (400 MHz, CDCl₃) δ 2.66 (tt, J=9.6, 3.9 Hz, 1H), 2.48 (dt, J=14.8, 5.4 Hz, 2H), 2.36 (m, 2H), 2.18 (ddd, J=14.1, 8.7, 4.4 Hz, 2H), 2.01 (dtd, J=14.4, 9.5, 4.8 Hz, 2H), 1.48 (s, 9H). LC/MS: m/z calculated 198.3, found 199.1 (M+1)+.

### Step C: Intermediate 9

### tert-butyl 4-(((trifluoromethyl)sulfonyl)oxy)cyclohex-3-enecarboxylate

To a solution of intermediate 8 (3 g, 15.1 mmol) in THF (60 mL) was added Li-HMDS (16.8 mL, 1M in THF, 16.8 mmol) at −78° C. The resulting mixture was stirred at -78° C. for 1 hr, followed by the addition of a solution of PhNTf₂ (6 g, 16.6 mmol) in THF (10 mL). The reaction mixture was warmed up to room temperature and stirred for 12 hr. The mixture was quenched with 1 M NaHSO₄ solution and extracted with EtOAc. The organic layer was dried over Na₂SO₄, filtered and concentrated under reduced pressure to give the crude product which was purified by silica gel chromatography (0-15% EtOAc/PE) to afford intermediate 9 (3.2 g, 64% yield) as a colorless oil. ¹H NMR (400 MHz, CDCl₃) δ 5.76 (dd, J=4.4, 1.7 Hz, 1H), 2.51 (ddd, J=13.1, 6.8, 3.1 Hz, 1H), 2.41 (m, 4H), 2.08 (m, 1H), 1.90 (m, 1H), 1.45 (s, 9H). LC/MS: m/z calculated 330.1, found 331.2 (M+1)+.

### Step D: Intermediate 10

### tert-butyl 4-(4,4,5,5-tetramethyl-1,3,2-dioxaborolan-2-yl)cyclohex-3-enecarboxylate

A mixture of intermediate 9 (9.1 g, 27.5 mmol), B₂Pin₂ (7.7 g, 30.4 mmol), Pd(dppf)Cl₂ (0.67 g, 0.82 mmol), dppf (0.46 g, 0.82 mmol) and KOAc (8.1 g, 83 mmol) in dioxane (90 mL) was stirring at 90° C. under N₂ atmosphere for 18 hr. The reaction mixture was partitioned between EtOAc and water. The layers were separated and the organic layer was washed with brine, dried over Na₂SO₄, filtered and concentrated under reduced pressure to give the crude product which was purified by silica gel chromatography (0-5% EtOAc/PE) to afford intermediate 10 (6.1 g, 72% yield) as a colorless oil. ¹H NMR (400 MHz, CDCl₃) δ 6.47 (d, J=2.0 Hz, 1H), 2.34 (m, 1H), 2.19 (m, 3H), 2.04 (m, 1H), 1.90 (m, 1H), 1.49 (m, 1H), 1.37 (s, 9H), 1.19 (s, 12H). LC/MS: m/z calculated 308.2, found 309.4 (M+1)+.

### Step E: Intermediate 11

### (4-(tert-butoxycarbonyl)cyclohex-1-en-1-yl)boronic acid

To s solution of intermediate 10 (1.38 g, 4.5 mmol) in acetone (16 mL) and H₂O (8 mL) was added NalO₄ (2.87 g, 13.4 mmol) and NH₄OAc (1 g, 13.4 mmol). The resulting mixture was stirred at room temperature for 3 hr and partitioned between EtOAc and water. The layers were separated and the organic layer was washed with brine, dried over Na₂SO₄, filtered and concentrated under reduced pressure to give the crude product which was triturated with PE to afford the product intermediate 11 (950 mg, 95% yield) as a white solid. ¹H NMR (400 MHz, CDCl₃) δ 6.91 (d, J=1.8 Hz, 1H), 2.41 (m, 4H), 2.16 (m, 1H), 2.00 (m, 1H), 1.61 (ddd, J=7.9, 7.0, 3.5 Hz, 1H), 1.46 (s, 9H). LC/MS: m/z calculated 226.1, found 227.3 (M+1)+.

Synthesis of the amino alcohol intermediates 24 was accomplished according to the following procedures.

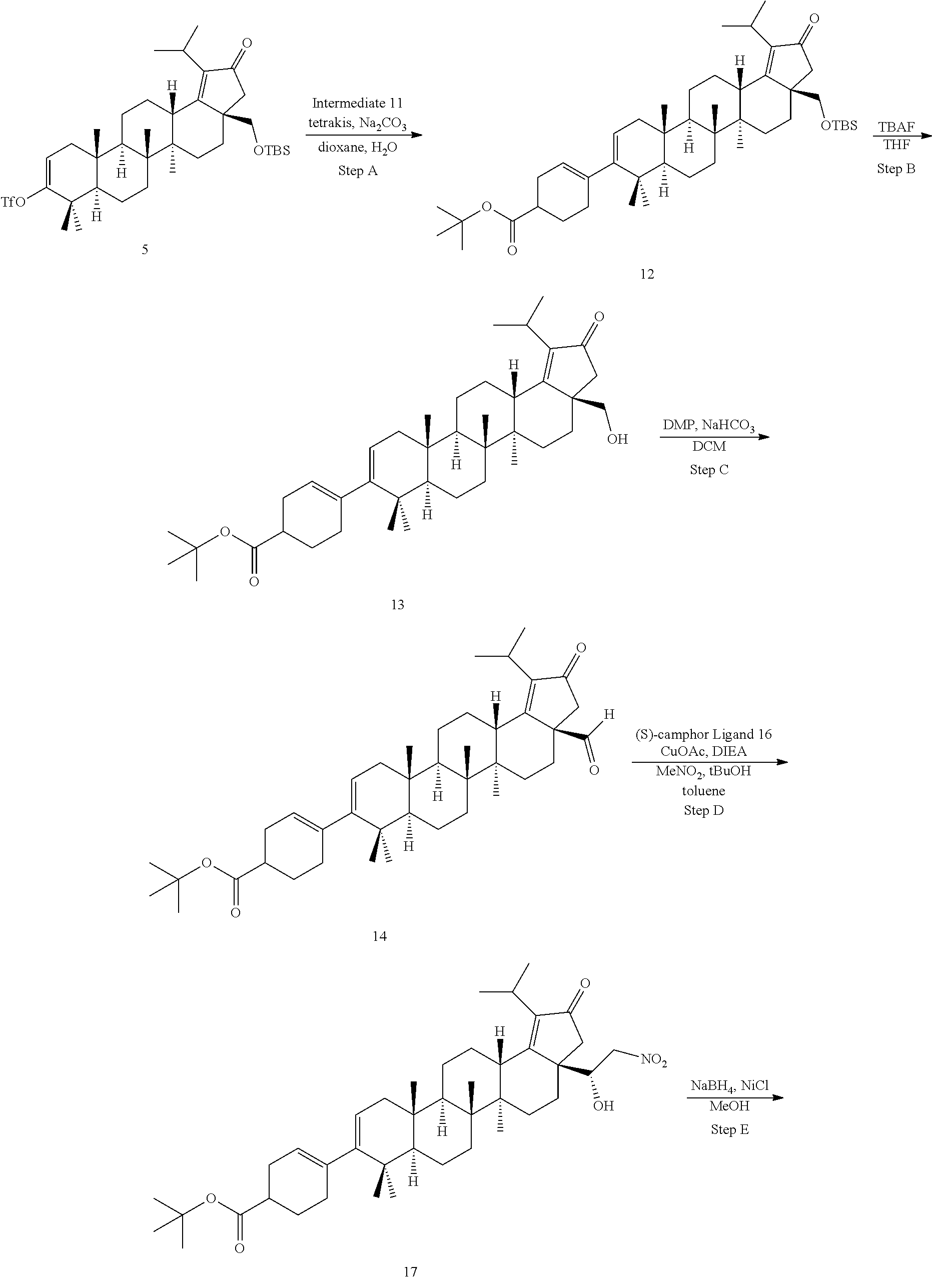

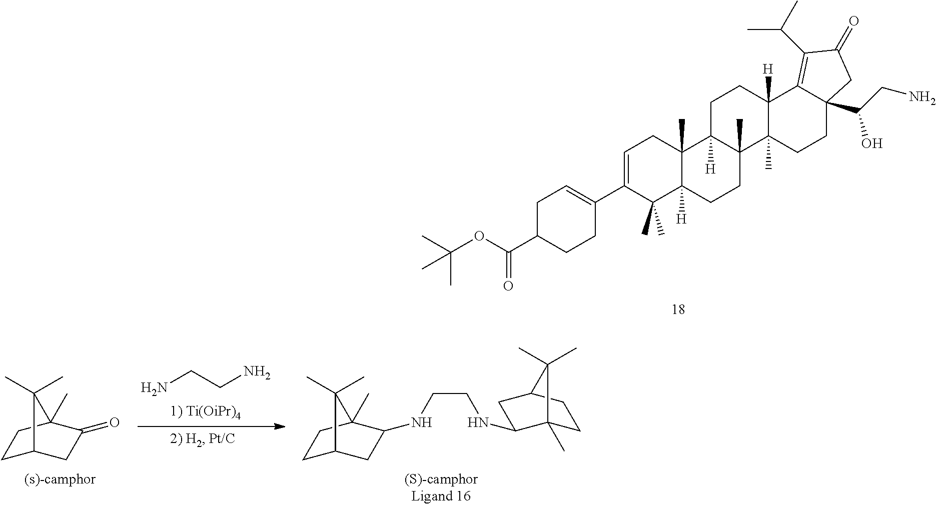

### Step A: Intermediate 12

### tert-butyl 4-((3aR,5aR, 5bR, 7aR,11aS,11bR, 13aS)-3a-(((tert-butyldimethylsilyl)oxy)methyl)-1-isopropyl-5a,5b,8,8,11a-penta methyl-2-oxo-3,3a,4,5,5a,5b,6,7,7a,8,11,11a, 11b,12,13,13a-hexadecahydro-2H-cyclopenta[a]chtysen-9-3/1)cyclohex-3-enecarboxylate

A mixture of intermediate 5 (7.7 g, 8.2 mmol), intermediate 11 (3.8 g, 12.3 mmol), tetrakis (1.9 g, 1.6 mmol) and Na₂CO₃ (2.61 g, 24.7 mmol) in dioxane (77 mL) and H₂O (19 mL) was stirred under N₂ atmosphere at 85° C. overnight. The resulting mixture was filtered and then partitioned between EtOAc and H₂O and layers were separated. The organic layer was washed with brine, dried over Na₂SO₄, filtered and concentrated under reduced pressure to give the crude product which was purified by silica gel chromatography (0-10% EtOAc/PE) to afford intermediate 12 (4.7 g, 78% yield) as a white solid. ¹H NMR (400 MHz, CDCl₃) δ 5.35 (s, 1H), 5.20 (d, J=6.0 Hz, 1H), 3.64 (dd, J=49.7, 9.5 Hz, 2H), 3.16 (m, 1H), 2.77 (dd, J=12.1, 3.6 Hz, 1H), 2.41 (m, 3H), 1.91 (m, 25H), 1.01 (m, 36H), 0.02 (d, J=1.6 Hz, 6H).

### Step B: Intermediate 13

### tert-butyl 4-((3aR,5aR,5bR,7aR,11aS,11bR,13aS)-3a-(hydroxymethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

To a solution of intermediate 12 (4.9 g, 6.7 mmol) in THF (26 mL) was added TBAF (13.3 mL, 1M in THF, 13.3 mmol). The reaction was stirred at room temperature overnight, then was partitioned between EtOAc and H₂O and the layers were separated. The organic layer was washed with brine, dried over Na₂SO₄, filtered and concentrated under reduced pressure to give the intermediate 13 (4.4 g, quant. yield) as a white solid which was used in the next step without further purification. LC/MS: m/z calculated 618.5, found 619.7 (M+1)+.

### Step C: Intermediate 14

### tert-butyl 4-((3aR,5aR,5bR,7aR,11aS,11bR,13aS)-3a-formyl-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

To a solution of intermediate 13 (4.4 g, 7.1 mmol) in DCM (44 mL) was added NaHCO₃ (6.0 g, 71 mmol) and DMP (6.0 g, 14.2 mmol). The reaction was stirred at room temperature for 4 h, then was diluted with DCM and washed with sat. Na₂S₂O₃ solution. The layers were separated and the organic layer was washed with brine, dried over Na₂SO₄, filtered and concentrated under reduced pressure. The residue was purified by silica gel chromatography (0-10% EtOAc/PE) to afford intermediate 14 (1.8 g, 41% yield) as a white solid. ¹H NMR (400 MHz, CDCl₃) δ 9.32 (d, J=1.2 Hz, 1H), 5.35 (s, 1H), 5.20 (d, J=5.9 Hz, 1H), 3.26 (dt, J=13.9, 7.0 Hz, 1H), 1.60 (m, 56H).

### Synthesis of (S) Camphor Derived Chiral Diamine Ligand 16

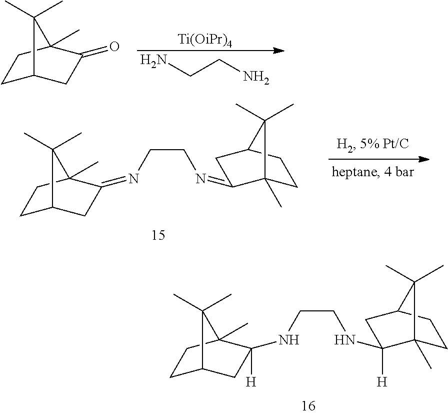

### Step A: Intermediate 15

### N,N′-bis(isobornyl)ethylenediimine

Titanium (IV) isopropoxide (235.4 g, 830 mmol, 1.04 eq) was added to a flask containing (1S)-(−)-camphor (121.43 g, 798 mmol, 1 eq) at ambient temperature. The reaction was then heated to ˜50° C. Next, ethylenediamine (31.2 g, 518 mmol, 0.65 eq) was charged to the reaction. The temperature was then kept above 45° C. during the addition. The reaction was then heated to ˜91° C. for 17 hours. Next, the reaction was cooled to 20-25° C. and heptane (1.2 L) was added. Water (29.9 g, 1659 mmol, 2.08 eq) was added over at least 15 minutes. The slurry was then stirred for 20 minutes at ambient temperature, cooled to ˜0° C., and stirred for 30 minutes at ˜0° C. The slurry was then filtered and the solids washed with heptane (607 mL). The diimine solution was stored ˜5° C. overnight. The solution was then warmed to ambient temperature and filtered to remove additional salts. Next, the solution was partially concentrated and filtered through Celite™. Finally, the solution was concentrated to ˜608 mL and used as is in the next reaction.

### Step B: Liciand 16

### N,N′-bis(isobornyl)ethylenediamine ligand

To a 1 L Jacketed Lab Reactor (JLR) was added the above diimine solution followed by 5% Pt/C (Johnson-Matthey, B501018-5, 6.6 g). The reaction was hydrogenated for ˜15 hours at 4 par at ambient temperature. The reaction was filtered and washed with heptane (300 mL). The solution was concentrated to provide a white solid (115.07 g). This two step procedure was repeated. Both batches were combined. Attempts to crystallize the material from i-PrOH and water failed. The product was extracted with heptane. The heptane layer was then washed with water, brine, dried over sodium sulfate, filtered and concentrated on rotovapor and then high vacuum. Ligand 16 (222.18 g) was obtained as a white solid and used is. ¹H NMR (500 MHz, CDCl₃) δ 2.69-2.61 (m, 1H), 2.53-2.47 (m, 2H), 1.71-1.63 (m, 2H), 1.6-1.43 (m, 3H), 1.1-1.01 (m, 2H), 1.01-0.98 (m, 3H), 0.89-0.83 (m, 3H), 0.81-0.78 (m, 3H).

### Step D: Intermediate 17

### tert-butyl 4-((3aR,5aR,5bR,7aR,11aS,11bR,13aS)-3a-((R)-1-hydroxy-2-nitroethyl)-1-isopropyl-5a,5b,8,8,11a-pentamethyl-2-oxo-3,3a,4,5,5a,5b,6,7,7a,8,11,11a, 11b,12,13,13a-hexadecahydro-2H-cyclopenta[a]chrysen-9-yl)cyclohex-3-enecarboxylate

A mixture of intermediate 14 (2.14 g, 3.5 mmol), ligand 16 (137 mg, 0.42 mmol) and CuOAc (42.5 mg, 0.35 mmol) in t-BuOH (21 mL) and toluene (7 mL) was stirred at room temperature. for 5 h. The resulting mixture was cooled to 5° C. and MeNO₂ (1.5 g, 24.3 mmol) and DIPEA (0.54 g, 4.2 mmol) was added. After stirred at 5° C. for 4 days, the reaction was diluted with MTBE and washed with 15% NH₄Cl solution, water, and brine. The organic layer was dried over Na₂SO₄, filtered and concentrated under reduced pressure to give a residue which was purified by silica gel chromatography (0-10% EtOAc/PE) to afford intermediate 17 (1.8 g, 76% yield) as a white solid. ¹H NMR (400 MHz, CDCl3) δ 5.29 (s, 1H), 5.13 (d, J=5.8 Hz, 1H), 4.79 (d, J=10.3 Hz, 1H), 4.07 (m, 2H), 3.10 (m, 1H), 2.20 (m, 13H), 1.14 (m, 44H). LC/MS: m/z calculated 677.5, found 678.8 (M+1)+.

### Step E: Intermediate 18

### tert-butyl 4-((3aR,5aR,5bR,7aR,11aS,11bR,13aS)-3a-((R)-2-amino-1-hydroxyethyl)-1-isopropyl-5a,5b,8,8,11a-pentamethyl-2-oxo-3,3a,4,5,5a,5b,6,7,7a,8,11,11a, 11b,12,13,13a-hexadecahydro-2H-cyclopenta[a]chrysen-9-yl)cyclohex-3-enecarboxylate

At 0° C., to a suspension of intermediate 17 (500 mg, 0.7 mmol) and NiCl₂.6H₂O (295 mg, 1.25 mmol) in MeOH (15 mL) was added NaBH₄ (310 mg, 8.2 mmol). After stirring at room temperature for 30 min, the resulting mixture was quenched with sat. NaHCO₃ solution and extracted with DCM. The layers were separated and the organic layer was washed with brine, dried over Na₂SO₄, filtered and concentrated under reduced pressure to give a residue which was purified by silica gel chromatography (0-10% MeOH/DCM) to give intermediate 18 (320 mg, 67% yield) as a grey solid. LC/MS: m/z calculated 647.5, found 649.1 (M+1)+.

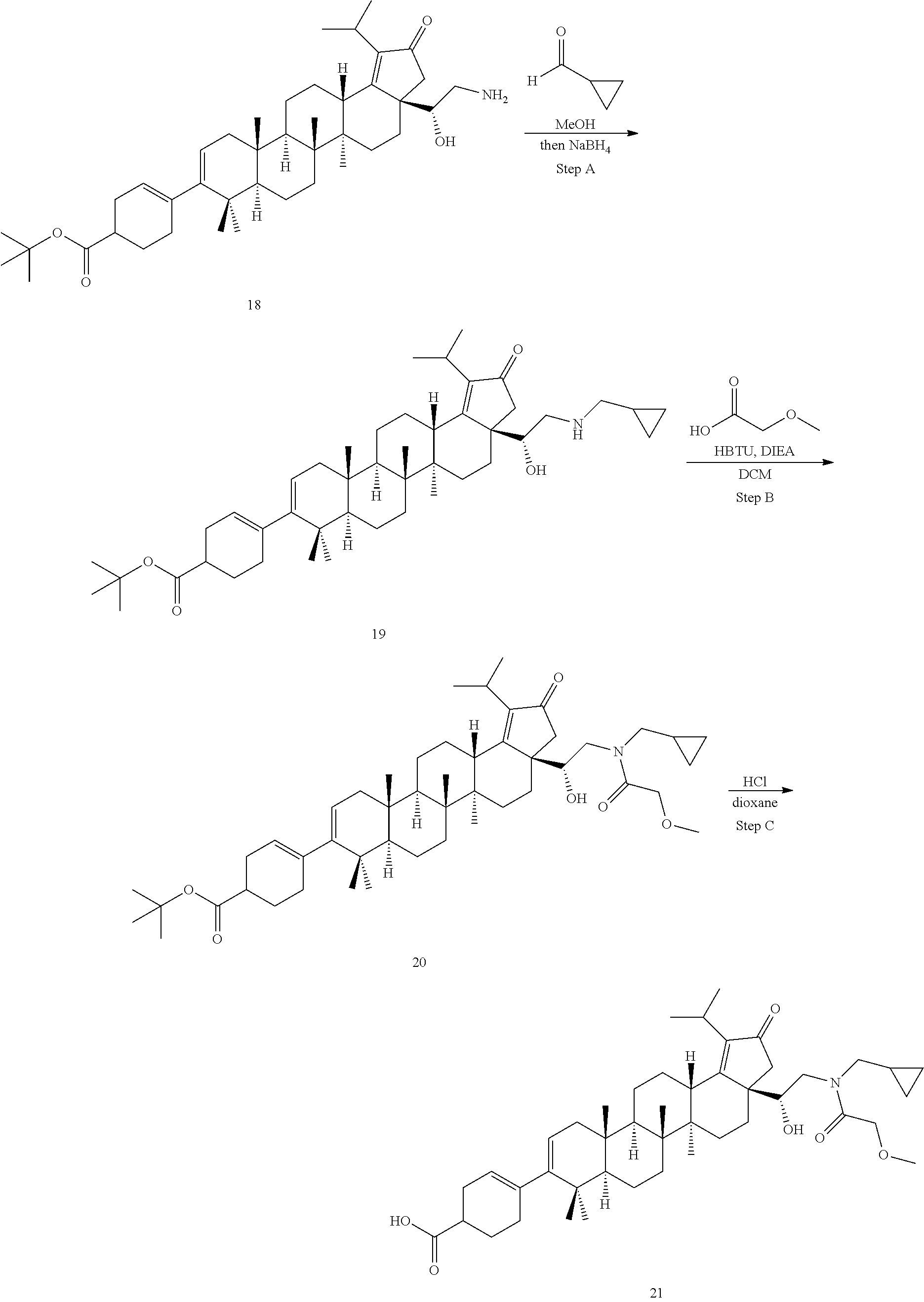

### Example 1: Compound 21

### 4-((3aR,5aR,5bR,7aR,11aS,11bR,13aS)-3a4(R)-2-(N-(cyclopropylmethyl)-2-methoxyacetamido)-1-hydroxyethyl)-1-isopropyl-5a, 5b,8,8,11a-pentamethyl-2-oxo-3,3a,4,5,5a,5b, 6,7,7a,8,11,11a, 11b,12,13,13a-hexadecahydro-2H-cyclopenta[a]chrysen-9-yl)cyclohex-3-enecarboxylic acid

### Step A: Intermediate 19

### tert-butyl 4-((3aR,5aR,5bR,7aR,11aS,11bR,13aS)-3a-((R)-2-((cyclopropylmethyl)amino)-1-hydroxyethyl)-1-isopropyl-5a,5b,8,8,11a-pentamethyl-2-oxo-3,3a,4,5,5a, 5b, 6,7,7a,8,11,11a,11b,12,13,13a-hexadecahydro-2H-cyclopenta[a]chrysen-9-yl)cyclohex-3-enecarboxylate

A mixture of intermediate 18 (250 mg, 0.38 mmol) and cyclopropanecarbaldehyde (32 mg, 0.45 mmol) in MeOH (10 mL) and DCE (a few drops) was stirred at room temperature for 2 hr. The resulting mixture was ice cooled and NaBH₄ (14.3 mg, 0.38 mmol) was added portionwise. After stirring at room temperature for 30 min, the reaction was quenched with sat. NH₄Cl solution and extracted with DCM. The organic layer was washed with brine, dried over Na₂SO₄, filtered and concentrated under reduced pressure to give a residue which was purified by silica gel chromatography (0-10% MeOH/DCM) to afford intermediate 19 (180 mg, 66% yield) as a white solid. ¹H NMR (400 MHz, CDCl₃) δ 5.35 (s, 1H), 5.20 (d, J=5.8 Hz, 1H), 4.10 (dd, J=10.8, 2.9 Hz, 1H), 3.15 (m, 1H), 1.68 (m, 63H), 0.49 (m, 2H), 0.12 (m, 2H). LC/MS: m/z calculated 701.5, found 702.8 (M+1)+.

### Step B: Intermediate 20

### tert-butyl 4-((3aR,5aR,5bR,7aR,11aS,11bR,13aS)-3a-((R)-2-(N-(cyclopropylmethyl)-2-methoxyacetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

A solution of intermediate 19 (30 mg, 0.044 mmol) and dimethylglycine (6 mg, 0.066 mmol) in DCM was added HBTU (33 mg, 0.086 mmol) and DIPEA (11 mg, 0.086 mmol). After stirred at room temperature for 1 hr, the resulting mixture was quenched with sat. NaHCO₃ solution and extracted with DCM. The organic layer was washed with brine, dried over Na₂SO₄, filtered and concentrated under reduced pressure to give a residue which was purified by silica gel chromatography (0-10% MeOH/DCM) to afford intermediate 20 (25 mg, 75% yield) as a white solid. ¹H NMR (400 MHz, CDCl₃) δ 5.35 (s, 1H), 5.20 (d, J=5.6 Hz, 1H), 4.33 (dd, J=35.1, 10.8 Hz, 1H), 4.15 (s, 2H), 3.79 (m, 1H), 3.16 (m, 8H), 1.69 (m, 57H), 0.61 (m, 2H), 0.19 (m, 2H).

### Step C: Compound 21

### 4-((3aR,5aR,5bR,7aR,11aS,11bR,13aS)-3a4(R)-2-(N-(cyclopropylmethyl)-2-methoxyacetamido)-1-hydroxyethyl)-1-isopropyl-5a, 5b,8,8,11a-pentamethyl-2-oxo-3,3a,4,5,5a,5b, 6,7,7a,8,11,11a,11b,12,13,13a-hexadecahydro-2H-cyclopenta[a]chrysen-9-yl)cyclohex-3-enecarboxylic acid

A mixture of intermediate 20 (22 mg, 0.028 mmoL) in 4N HCl in dioxanes (1 mL) was stirred at room temperature overnight. The reaction was concentrated under reduced pressure to give a residue that was purified by reverse phase chromatography (30-100% ACN/H₂O+0.1% FA) to give compound 21 (10 mg, 49%) as a white powder. ¹H NMR (400 MHz, CDCl₃) δ 5.84 (s, 1H), 5.40 (s, 1H), 5.23 (d, J=6.4 Hz, 1H), 4.12 (m, 3H), 3.46 (s, 3H), 3.19 (m, 1H), 1.71 (m, 53H), 0.59 (m, 2H), 0.17 (m, 2H); LC/MS: m/z calculated 717.5, found 718.8 (M+1)⁺.

### Example 2: Compound 22

### 4-((3aR,5aR,5bR,7aR,11aS,11bR,13aS)-3a4(R)-2-(N-(cyclopropylmethyl)-2-(dimethylamino)acetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid

The title compound made in a similar manner to example 1 and was isolated (10 mg, 43%) as a white powder. 1H NMR (400 MHz, MeOD) δ 5.36 (s, 1H), 5.22 (d, J=6.2 Hz, 1H), 4.43 (m, 2H), 3.77 (m, 4H), 3.16 (m, 3H), 2.70 (m, 8H), 2.41 (m, 1H), 1.55 (m, 44H), 0.60 (m, 2H), 0.26 (m, 2H); LC/MS: m/z calculated 730.5, found 732.1 (M+1)⁺.

### Example 3: Compound 23

4-((3aR,5aR,5bR,7aR,11aS,11bR,13aS)-3a-((R)-2-(N-(cyclopropylmethyl)-2-(pyrrolidin-1-yl)acetamido)-1-hydroxyethyl)-1-isopropyl-5a,5b,8,8,11a-pentamethyl-2-oxo-3,3a,4,5,5a,5b, 6,7,7a,8,11,11a,11b,12,13,13a-hexadecahydro-2H-cyclopenta[a]chrysen-9-yl)cyclohex-3-enecarboxylic acid

The title compound made in a similar manner to example 1 and was isolated (12 mg, 46%) as a white powder. ¹H NMR (400 MHz, MeOD) δ 5.36 (s, 1H), 5.22 (d, J=6.1 Hz, 1H), 4.31 (m, 2H), 4.01 (m, 2H), 3.54 (m, 1H), 3.18 (m, 6H), 1.89 (m, 53H), 0.57 (m, 2H), 0.25 (m, 2H); LC/MS: m/z calculated 756.5, found 757.6 (M+1)⁺.

### Example 4: Compound 24

### 4-((3aR,5aR,5bR,7aR,11aS,11bR,13aS)-3a-((R)-2-(N-(cyclopropylmethyl)-2-(2-oxopyrrolidin-1-yl)acetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid

The title compound made in a similar manner to example 1 and was isolated (19 mg, 62%) as a white powder. ¹H NMR (400 MHz, CDCl₃) δ 8.78 (br, 1H), 6.12 (dd, J=19.6, 9.8 Hz, 1H), 5.38 (s, 1H), 5.16 (dd, J=12.0, 5.4 Hz, 1H), 4.27 (m, 2H), 3.77 (m, 1H), 3.62 (m, 2H), 3.22 (m, 1H), 1.98 (m, 56H), 0.66 (d, J=7.6 Hz, 2H), 0.47 (m, 2H); LC/MS: m/z calculated 770.5, found 771.9 (M+1)⁺.

### Example 5: Compound 25

### 4-((3aR,5aR, 5bR,7aR,11aS, 11bR, 13aS)-3a-((R)-2-(N-(cyclobutyl methyl)-2-(dimethylamino)acetamido)-1-hydroxyethyl)-1-isopropyl-5a,5b,8,8,11a-pentamethyl-2-oxo-3,3a, 4,5,5a,5b, 6,7,7a,8,11,11a,11b,12,13,13a-hexadecahydro-2H-cyclopenta[a]chrysen-9-yl)cyclohex-3-enecarboxylic acid

The title compound made in a similar manner to example 1 and was isolated (7 mg, 38%) as a white powder. ¹H NMR (400 MHz, MeOD) δ 5.36 (s, 1H), 5.22 (m, 1H), 4.12 (m, 5H), 3.44 (d, J=7.4 Hz, 2H), 3.21 (m, 2H), 2.71 (m, 11H), 1.61 (m, 50H); LC/MS: m/z calculated 744.5, found 746.0 (M+1)⁺.

### Example 6: Compound 26

### 4-((3aR,5aR,5bR,7aR,11aS,11bR,13aS)-3a-((R)-2-(N-(cyclobutylmethyl)-2-methoxyacetamido)-1-hydroxyethyl)-1-isopropyl-5a,5b,8,8,11a-pentamethyl-2-oxo-3,3a,4,5,5a,5b 6,7,7a,8,11,11a,11b,12,13,13a-hexadecahydro-2H-cyclopenta[a]chrysen-9-yl)cyclohex-3-enecarboxylic acid

The title compound made in a similar manner to example 1 and was isolated (10 mg, 43%) as a white powder. ¹H NMR (400 MHz, MeOD) δ 5.36 (s, 1H), 5.22 (d, J=5.7 Hz, 1H), 4.22 (m, 3H), 3.43 (d, J=20.7 Hz, 3H), 2.06 (m, 60H); LC/MS: m/z calculated 731.5, 732.8 found (M+1)⁺.

### Example 7: Compound 27

### 4-((3aR,5aR,5bR,7aR,11aS,11bR,13aS)-3a-((R)-2-(N-(cyclobutylmethyl)-2-(pyrrolidin-1-yl)acetamido)-1-hydroxyethyl)-1-isopropyl-5a, 5b,8,8,11a-pentamethyl-2-oxo-3,3a,4,5,5a,5b, 6,7,7a,8,11,11a,11b,12,13,13a-hexadecahydro-2H-cyclopenta[a]chrysen-9-yl)cyclohex-3-enecarboxylic acid

The title compound made in a similar manner to example 1 and was isolated (18 mg, 55%) as a white powder. ¹H NMR (400 MHz, MeOD) δ 5.36 (s, 1H), 5.22 (m, 1H), 4.22 (m, 5H), 3.44 (m, 2H), 3.17 (m, 3H), 1.09 (m, 60H). LC/MS: m/z calculated 770.6, 771.9 found (M+1)⁺.

### Example 8: Compound 28

### 4-((3aR,5aR,5bR,7aR,11aS,11bR,13aS)-3a-((R)-2-(4-chlorobenzyl)amino)-1-hydroxyethyl)-1-isopropyl-5a,5b, 8,8,11a-pentamethyl-2-oxo-3,3a,4,5,5a,5b, 6,7,7a,8,11,11a,11b,12,13,13a-hexadecahydro-2H-cyclopenta[a]chrysen-9-yl)cyclohex-3-enecarboxylic acid

The title compound made in a similar manner example 1. In step A: 4-chlorobenzaldehyde, NaBH₃CN, and THF were used to give tert-butyl 4-((3aR,5aR,5bR,7aR,11aS,11bR,13aS)-3a-((R)-2-((4-chlorobenzyl)amino)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate. A solution of tert-butyl 4-((3aR,5aR,5bR,7aR,11aS,11bR,13aS)-3a-((R)-2-((4-chlorobenzyl)amino)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate (8.0 mg, 0.01 mmol) in DCM (1 mL) was treated by the addition of TFA (0.2 mL, 2.69 mmol). The reaction was stirred at room temperature for 30 minutes and the reaction was washed with sat. NaHCO₃ and brine. The organic layer was dried over Na₂SO₄, filtered, and concentrated to give a residue which was purified by reverse phase chromatography (30-100% ACN/H₂O+0.1% FA) to afford compound 28 (4.4 mg, 61%) as a white powder. ¹H NMR (400 MHz, CDCl₃) δ 7.34 (d, J=8.4 Hz, 2H), 7.28 (m, 2H), 5.40 (s, 1H), 5.24 (d, J=5.9 Hz, 1H), 4.23 (d, J=8.7 Hz, 1H), 3.85 (d, J=9.1 Hz, 2H), 3.42 (m, 1H), 3.11 (m, 1H), 2.42 (m, 7H), 1.47 (m, 44H); LC/MS: m/z calculated 715.4, found 716.5 (M+1)⁺.

### Example 9: Compound 29

The title compound made in a similar manner to example 1. In step A: 2-(dimethylamino)acetaldehyde, NaBH₃CN, and THF were used. In step B a reductive amination using 4-chlorobenzaldehyde, NaBH₃CN, and THF were used to give tert-butyl 4-((3aR,5aR,5bR,7aR,11aS,11bR,13aS)-3a-((R)-2-((4-chlorobenzyl)(2-(dimethylamino)ethyl)amino)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate. A solution of tert-butyl 4-((3aR,5aR,5bR,7aR,11aS,11bR,13aS)-3a-((R)-2-((4-chlorobenzyl)(2-(dimethylamino)ethyl)amino)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate (34 mg, 0.04 mmol) in DCM (2 mL) was treated with TFA (0.4 mL, 5.3 mmol) and stirred for 30 min at room temperature. The reaction was washed with sat. NaHCO₃ and brine. The organic layer was dried over Na₂SO₄, filtered, and concentrated to give a residue which was purified by reverse phase chromatography (30-100% ACN/H₂O+0.1% FA) to afford a residue that was treated with a few drops of HCl in dioxanes to give compound 29 (16 mg, 51%) as a the dihydrochloride salt. ¹H NMR (400 MHz, CDCl₃) δ 7.31 (m, 4H), 5.37 (s, 1H), 5.21 (d, J=4.9 Hz, 1H), 4.06 (d, J=9.4 Hz, 1H), 3.61 (d, J=9.4 Hz, 1H), 3.61 (s, 2H), 3.40 (s, 1H), 3.10 (m, 1H), 1.63 (m, 59H). LC/MS: m/z calculated 786.5, found 787.0 (M+1)⁺.

Synthesis of the amino alcohol intermediate 34 was accomplished according to the following procedures.

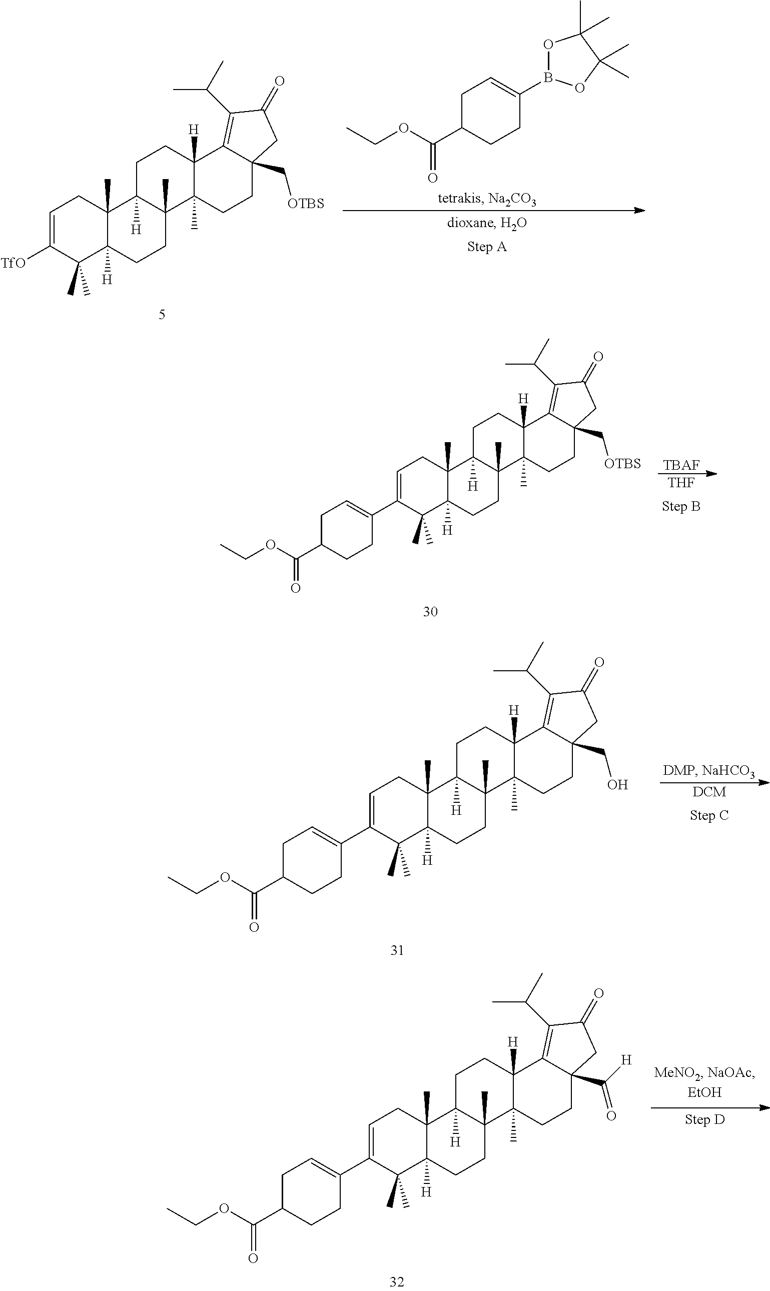

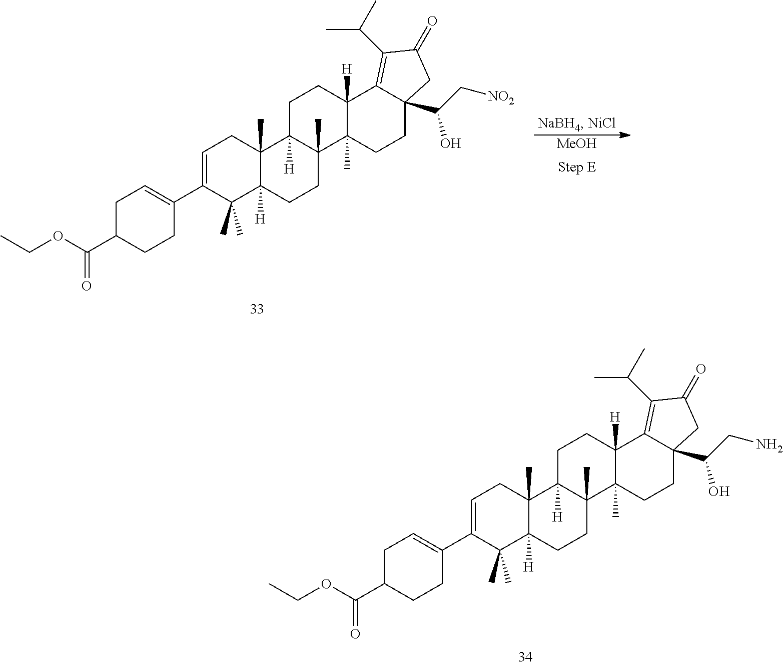

### Step A: Intermediate 30

### Ethyl 4-((3aR,5aR,5bR,7aR,11aS,11bR,13aS)-3a-(((tert-butyldimethylsilyl)oxy)methyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

A solution of intermediate 5 (1 g, 1.43 mmol) and ethyl 4-(4,4,5,5-tetramethyl-1,3,2-dioxaborolan-2-yl)cyclohex-3-enecarboxylate (0.80 g, 2.86 mmol) were treated and in a similar manner to step A, intermediate 12 to give intermediate 30 (0.77 g, 76.7%) as a white solid. ¹H NMR (400 MHz, CDCl₃) δ 5.36 (s, 1H), 5.21 (d, J=5.9 Hz, 1H), 4.13 (m, 2H), 3.64 (dd, J=67.8, 9.5 Hz, 2H), 3.16 (m, 1H), 2.77 (m, 1H), 2.06 (m, 32H), 1.01 (m, 17H), 0.84 (s, 9H), 0.02 (d, J=1.7 Hz, 6H).

### Step B: Intermediate 31

### Ethyl 4-((3aR,5aR,5bR,7aR,11aS,11bR,13aS)-3a-(hydroxymethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

Intermediate 30 (0.87 g, 1.23 mmol) was treated with TBAF (2.46 mL, 2.46 mmol) in a similar manner to step B, intermediate 13 to give a residue that was purified by silica gel chromatography (0-10% EtOAc/PE) to give intermediate 31 (0.36 g, 49.5%) as a white solid. ¹H NMR (400 MHz, CDCl₃) δ 5.37 (s, 1H), 5.22 (d, J=5.9 Hz, 1H), 4.15 (q, J=7.1 Hz, 2H), 3.72 (dd, J=24.4, 10.2 Hz, 2H), 3.21 (dt, J=13.9, 7.0 Hz, 1H), 2.81 (dd, J=12.6, 3.2 Hz, 1H), 2.50 (m, 2H), 1.82 (m, 23H), 1.12 (m, 25H).

### Step C: Intermediate 32

### Ethyl 4-((3aR,5aR, 5bR,7aR,11aS,11bR,13aS)-3a-formyl-1-isopropyl-5a, 5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

Intermediated 31 (0.82 g, 1.38 mmol) was treated with DMP (1.17 g, 2.76 mmol) in a similar manner to step C, intermediate 14 to give intermediate 32 (0.41 g, 49.9%) as a white solid. ¹H NMR (400 MHz, CDCl₃) δ 9.32 (d, J=1.1 Hz, 1H), 5.36 (s, 1H), 5.20 (d, J=5.9 Hz, 1H), 4.14 (q, J=7.1 Hz, 2H), 3.26 (dt, J=13.9, 7.0 Hz, 1H), 2.53 (m, 2H), 2.01 (m, 13H), 1.22 (m, 35H).

### Step D: Intermediate 33

### Ethyl 4-((3aR,5aR,5bR,7aR,11aS,11bR,13aS)-3a-((R)-1-hydroxy-2-nitroethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

A suspension of intermediate 32 (523 mg, 0.89 mmol) and NaOAc (0.109 g, 1.33 mmol) in EtOH (25 mL) and MeNO₂ (25 mL) was stirred for 48 hours. The solution was partitioned between EtOAc and water. The organic layer was washed with brine, dried over Na2SO4, filtered, and concentrated under reduced pressure to give a residue that was purified by silica gel chromatography (0-30% EtOAc, PE) to give intermediate 33 (0.48 g, 83%) as a white solid as a mixture of diastereoisomers with -5:1 ratio. ¹H NMR (400 MHz, CDCl₃) δ 5.37 (s, 1H), 5.21 (d, J=5.9 Hz, 1H), 4.86 (m, 1H), 4.15 (m, 3H), 3.49 (d, J=5.2 Hz, 1H), 3.18 (dd, J=14.9, 7.9 Hz, 1H), 2.15 (m, 16H), 1.22 (m, 35H).

### Step E: Intermediate 34

### Ethyl 4-((3aR,5aR,5bR,7aR,11aS,11bR,13aS)-3a-((R)-2-amino-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylate

A solution of intermediate 33 (0.18 g, 0.28 mmol) was treated with NiCl₂ (0.097 g, 0.41 mmol) and NaBH₄ (0.10 g, 2.75 mmol) in a similar manner to step E, intermediate 18 to give intermediate 34 (0.146 g, 85%). LC/MS: m/z calculated 619.5, found 620.7 (M+1)⁺.

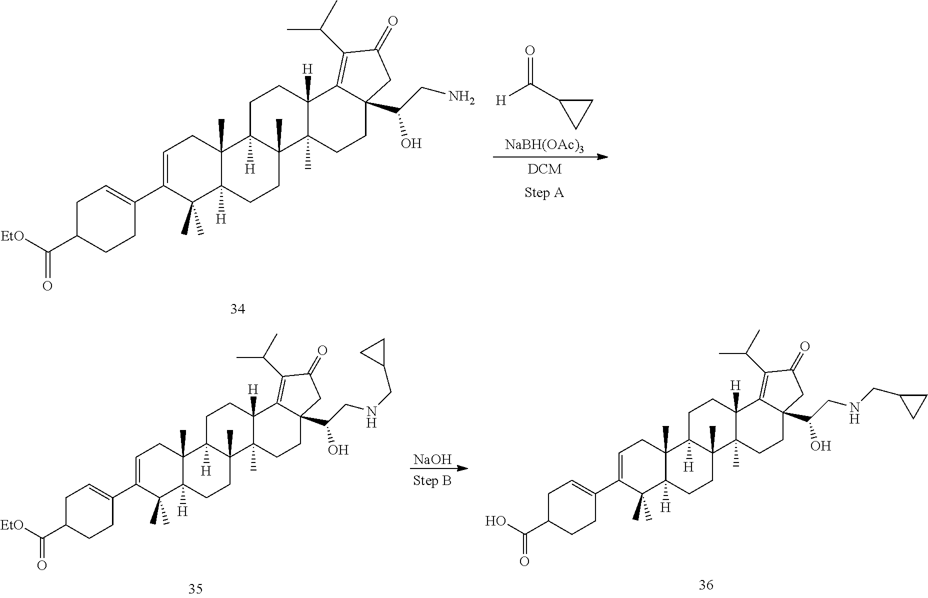

### Example 10: Compound 36

### 4-((3aR,5aR,5bR,7aR,11aS,11bR,13aS)-3a-((R)-2-((cyclopropylmethyl)amino)-1-hydroxyethyl)-1-isopropyl-5a,5b,8,8,11a-pentamethyl-2-oxo-3,3a,4,5,5a,5b 6,7,7a,8,11,11a,11b,12,13,13a-hexadecahydro-2H-cyclopenta[a]chrysen-9-yl)cyclohex-3-enecarboxylic acid

### Step A: Intermediate 35

### Ethyl 4-((3aR,5aR,5bR,7aR,11aS,11bR,13aS)-3a-((R)-2-((cyclopropyl methyl)amino)-1-hydroxyethyl)-1-isopropyl-5a,5b, 8,8,11a-pentamethyl-2-oxo-3,3a,4,5,5a,5b, 6,7,7a,8,11,11a,11b,12,13,13a-hexadecahydro-2H-cyclopenta[a]chrysen-9-yl)cyclohex-3-enecarboxylate

A solution of intermediate 34 (150 mg, 0.24 mmol) and cyclopropanecarbaldehyde (25 mg, 0.36 mmoL) in DCM (1 mL) was treated with NaBH(OAc)₃ (150 mg, 0.71 mmol). After stirring at room temperature overnight, the resulting mixture was quenched with sat. NaHCO₃ and extracted with DCM. The organic layer was washed with brine, dried over Na₂SO₄, filtered, and concentrated to a residue that was purified by silica gel chromatography (0-10% MeOH/DCM) to give intermediate 35 (58 mg, 36%). LC/MS: m/z calculated 673.5, found 674.8 (M+1)⁺.

### Step B: Compound 36

### 4-((3aR,5aR,5bR,7aR,11aS,11bR,13aS)-3a-((R)-2-((cyclopropylmethyl)amino)-1-hydroxyethyl)-1-isopropyl-5a,5b,8,8,11a-pentamethyl-2-oxo-3,3a,4,5,5a,5b, 6,7,7a,8,11,11a,11b,12,13,13a-hexadecahydro-2H-cyclopenta[a]chrysen-9-yl)cyclohex-3-enecarboxylic acid

A solution of intermediate 35 (25 mg, 0.037 mmol) in THF (1 mL) was treated with 1N NaOH (1 mL) and the reaction was heated at 80° C. overnight. The reaction was then acidified with 1N HCl to pH 3-4 and the extracted with DCM. The combine organics were washed with brine, dried over Na₂SO₄, filtered and concentrated to a residue that was purified by reverse phase chromatography (50-100% ACN/H₂O+0.1% FA) to give compound 36 (8 mg) as a white powder. ¹H NMR (400 MHz, DMSO) δ 5.30 (s, 1H), 5.17 (d, J=6.0 Hz, 1H), 4.23 (d, J=9.9 Hz, 1H), 3.12 (m, 2H), 2.70 (m, 9H), 1.52 (m, 45H), 0.50 (m, 2H), 0.29 (m, 2H); LC/MS: m/z calculated 645.5, found 647.0 (M+1)⁺.

### Administration and Formulation

In another embodiment, there is provided a pharmaceutical composition comprising a pharmaceutically acceptable diluent and a therapeutically effective amount of a compound of Formula I or a pharmaceutically acceptable salt thereof.

The compounds of the present invention can be supplied in the form of a pharmaceutically acceptable salt. The terms “pharmaceutically acceptable salt” refer to salts prepared from pharmaceutically acceptable inorganic and organic acids and bases. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds according to Formula I may be prepared. These pharmaceutically acceptable salts may be prepared in situ during the final isolation and purification of the compound, or by separately reacting the purified compound in its free acid or free base form with a suitable base or acid, respectively.

Illustrative pharmaceutically acceptable acid salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Preferred pharmaceutically acceptable salts include the salts of hydrochloric acid and trifluoroacetic acid.

Illustrative pharmaceutically acceptable inorganic base salts of the compounds of the present invention include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary base salts include aluminum, calcium, lithium, magnesium, potassium, sodium and zinc. Other exemplary base salts include the ammonium, calcium, magnesium, potassium, and sodium salts. Still other exemplary base salts include, for example, hydroxides, carbonates, hydrides, and alkoxides including NaOH, KOH, Na₂CO₃, K₂CO₃, NaH, and potassium-t-butoxide.

Salts derived from pharmaceutically acceptable organic non-toxic bases include salts of primary, secondary, and tertiary amines, including in part, trimethylamine, diethylamine, N, N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionisation in the salt may vary from completely ionised to almost non-ionised. Lists of suitable salts are found in Remington's Pharmaceutical Sciences, 17th ed., Mack Publishing Company, Easton, Pa., 1985, p. 1418, the disclosure of which is hereby incorporated by reference only with regards to the lists of suitable salts.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

Compounds of Formula I containing one or more asymmetric carbon atoms can exist as two or more stereoisomers. Where a compound of Formula I contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formula I, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula I contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of Formula I wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds of Formula I can generally be prepared by conventional techniques known to those skilled in the art using an appropriate isotopically-labelled reagents in place of the non-labelled reagent previously employed.

The compounds of the present invention may be administered as prodrugs. Thus, certain derivatives of compounds of Formula I, which may have little or no pharmacological activity themselves can, when administered into or onto the body, be converted into compounds of Formula I as ‘prodrugs’.

Administration of the chemical entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used the route and form of administration, and other factors. The drug can be administered more than once a day, such as once or twice a day.

Therapeutically effective amounts of the chemical entities described herein may range from approximately 0.01 to 200 mg per kilogram body weight of the recipient per day; such as about 0.01-100 mg/kg/day, for example, from about 0.1 to 50 mg/kg/day. Thus, for administration to a 70 kg person, the dosage range may be about 7-3500 mg per day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %.

### Example 11

### MT4 Cell Antiviral Assay

Experimental Procedure:

Antiviral HIV activity and compound-induced cytotoxicity were measured in parallel by means of a propidium iodide based procedure in the human T-cell lymphotropic virus transformed cell line MT4. Aliquots of the test compounds were serially diluted in medium (RPMI 1640, 10% fetal calf serum (FCS), and gentamycin) in 96-well plates (Costar 3598) using a Cetus Pro/Pette. Exponentially growing MT4 cells were harvested and centrifuged at 1000 rpm for 10 min in a Jouan centrifuge (model CR4 12). Cell pellets were resuspended in fresh medium (RPMI 1640, 20% FCS, 20% IL-2, and gentamycin) to a density of 5×105 cells/ml. Cell aliquots were infected by the addition of HIV-1 (strain IIIB) diluted to give a viral multiplicity of infection of 100×TCID50. A similar cell aliquot was diluted with medium to provide a mock-infected control. Cell infection was allowed to proceed for 1 hr at 37° C. in a tissue culture incubator with humidified 5% CO₂ atmosphere. After the 1 hr incubation the virus/cell suspensions were diluted 6-fold with fresh medium, and 125 μl of the cell suspension was added to each well of the plate containing pre-diluted compound. Plates were then placed in a tissue culture incubator with humidified 5% CO₂ for 5 days. At the end of the incubation period, cell number and hence HIV-induced cytopathy was estimated by either (A) propidium iodide staining, or by an (B) MTS tetrazolium staining method.

For propidium iodide readout, 27 μl of 5% Nonidet-40 was added to each well of the incubation plate. After thorough mixing with a Costar multitip pipetter, 60 μl of the mixture was transferred to filter-bottomed 96-well plates. The plates were analyzed in an automated assay instrument (Screen Machine, Idexx Laboratories). The control and standard used was 3′-azido-3′-deoxythymidine tested over a concentration range of 0.01 to 1 μM in every assay. The expected range of IC₅₀ values for 3′-azido-3′-deoxythymidine is 0.04 to 0.12 μM. The assay makes use of a propidium iodide dye to estimate the DNA content of each well.

For MTS readout, 20 μl CellTiter 96 AQ One Solution reagent (Promega #G3582) was added to each well. At 75 minutes following the addition of MTS reagent, absobance was read at 492 nM using a Tecan Sunrise 96-well plate reader.

Analysis:

The antiviral effect of a test compound is reported as an EC₅₀, i.e. the inhibitory concentration that would produce a 50% decrease in the HIV-induced cytopathic effect. This effect is measured by the amount of test compound required to restore 50% of the cell growth of HIV-infected MT4 cells, compared to uninfected MT4 cell controls. IC₅₀ was calculated by RoboSage, Automated Curve Fitting Program, version 5.00, 10 Jul. 1995.

For each assay plate, the results (relative fluorescence units, rfU, or OD values) of wells containing uninfected cells or infected cells with no compound were averaged, respectively. For measurements of compound-induced cytotoxicty, results from wells containing various compound concentrations and uninfected cells were compared to the average of uninfected cells without compound treatment. Percent of cells remaining is determined by the following formula:

Percent of cells remaining=(compound-treated uninfected cells, rfU, or OD values/untreated uninfected cells)×100.

A level of percent of cells remaining of 79% or less indicates a significant level of direct compound-induced cytotoxicity for the compound at that concentration. When this condition occurs the results from the compound-treated infected wells at this concentration are not included in the calculation of EC₅₀.

For measurements of compound antiviral activity, results from wells containing various compound concentrations and infected cells are compared to the average of uninfected and infected cells without compound treatment. Percent inhibition of virus is determined by the following formula:

Percent inhibition of virus=(1−((ave. untreated uninfected cells−treated infected cells)/(ave. untreated uninfected cells−ave. untreated infected cells)))×100.

Results:

Compounds of the present invention have anti-HIV activity in the range EC₅₀=1-1000 nM.

TABLE 4

| Example number | EC₅₀ NL4-3 wt (nM) | EC₅₀V370A (nM) |
|---|---|---|
| 1 | 7.9 | 8.9 |
| 2 | 5.4 | 11.2 |
| 3 | 3.3 | 3.7 |
| 4 | 7.1 | 20.9 |
| 5 | N/A | N/A |
| 6 | 10.0 | 20.4 |
| 7 | 5.8 | 4.1 |
| 8 | N/A | N/A |
| 9 | 6.8 | 10.7 |
| 10 | 9.3 | 25118.9 |

Table 4 shows EC₅₀ values for representative compounds of Table 2 after the HIV MT4 Antiviral Cell Assay of Example 11.

1-73. (canceled)

74. A compound having the structure of Formula I: 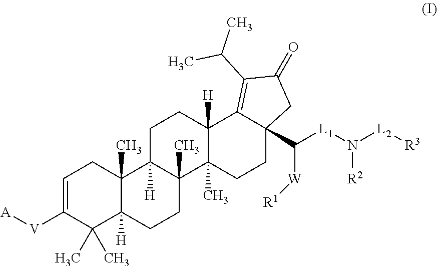
or a pharmaceutically acceptable salt thereof, wherein:
L₁ and L₂ are both (—CH₂—);
W is O;
R¹ is —H;
R² is selected from the group consisting of —(CH₂)ᵣNR⁷R⁸ and —C(O)R⁵;
R³ is selected from the group consisting of 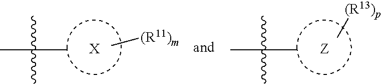
wherein:
X is phenyl,
Z is selected from the group consisting of cyclopropyl and cyclobutyl;
R⁵ is selected from the group consisting of —(CH₂)ᵣNR⁷R⁸, and —(CH₂)ᵣOR⁷;
R⁷ and R⁸ are independently selected from the group consisting of methyl, wherein R⁷ and R⁸ can be taken together with the nitrogen to which they are joined to form a pyrrolidine ring or 2-pyrrolidone ring;
R¹¹ and R¹³ are independently selected from the group consisting of chloro, bromo, and fluoro;
V is selected from the group consisting of —(C₄-C₈)cycloalkenyl, wherein:
V may be substituted with A², wherein:
A² is selected from the group consisting of —H or —CH₂OH, and —CH₂CH₂OH;
A is —COOH; 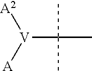
is selected from the group consisting of the following structures: 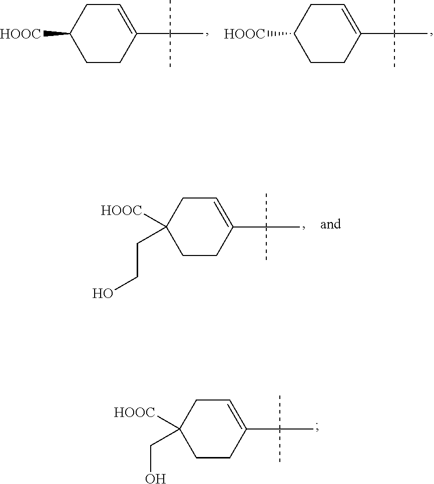
m is 0, 1, or 2; and
p is 0, 1, or 2;
r is 1, 2, or 3.

75. A compound or pharmaceutically acceptable salt therefore selected from the group consisting of example (1) 4-((3aR,5aR,5bR,7aR,11aS,11bR,13aS)-3a-((R)-2-(N-(cyclopropylmethyl)-2-methoxyacetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid, example (2) 4-((3aR,5aR,5bR,7aR,11aS,11bR,13aS)-3a-((R)-2-(N-(cyclopropylmethyl)-2-(dimethylamino)acetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid, example (3) 4-((3aR,5aR,5bR,7aR,11aS,11bR,13aS)-3a-((R)-2-(N-(cyclopropylmethyl)-2-(pyrrolidin-1-yl)acetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid, example (4) 4-((3aR,5aR,5bR,7aR,11aS,11bR,13aS)-3a-((R)-2-(N-(cyclopropylmethyl)-2-(2-oxopyrrolidin-1-yl)acetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid, example (5) 4-((3aR,5aR,5bR,7aR,11aS,11bR,13aS)-3a-((R)-2-(N-(cyclobutylmethyl)-2-(dimethylamino)acetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid, example (6) 4-((3aR,5aR,5bR,7aR,11aS,11bR,13aS)-3a-((R)-2-(N-(cyclobutylmethyl)-2-methoxyacetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid, example (7) 4-((3aR,5aR,5bR,7aR,11aS,11bR,13aS)-3a-((R)-2-(N-(cyclobutylmethyl)-2-(pyrrolidin-1-yl)acetamido)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid, example (8) 4-((3aR,5aR,5bR,7aR,11aS,11bR,13aS)-3a-((R)-2-(4-chlorobenzyl)amino)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid, example (9) 4-((3aR,5aR,5bR,7aR,11aS,11bR,13aS)-3a-((R)-2-((4-chlorobenzyl)(2-(dimethylamino)ethyl)amino)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid dihydrochloride, example (10) 4-((3aR,5aR,5bR,7aR,11aS,11bR,13aS)-3a-((R)-2-((cyclopropylmethyl)amino)-1-hydroxyethyl)-1-isopropyl-5a,5b,8,8,11a-pentamethyl-2-oxo-3,3a,4,5,5a,5b,6,7,7a,8,11,11a,11b,12,13,13a-hexadecahydro-2H-cyclopenta[a]chrysen-9-yl)cyclohex-3-enecarboxylic acid.

76. A pharmaceutical composition comprising a compound of claim 75, or a pharmaceutically acceptable salt thereof, and a pharmaceutically acceptable excipient.

77. The composition of claim 76, wherein the compound is present in an amorphous form.

78. The composition of claim 76, wherein the composition is in a tablet form.

79-80. (canceled)

81. A method of treating an HIV infection in a subject comprising administering to the subject a pharmaceutical composition according to claim 76.

82-83. (canceled)

84. The method of claim 76, further comprising administration of one or more additional agents active against HIV.

85. The method of claim 84, wherein said one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, GSK1349572, GSK1265744, vicriviroc (Sch-C), Sch-D, TAK779, maraviroc, TAK449, didanosine, tenofovir, lopinavir, and darunavir.

86. The method of claim 76, further comprising administration of one or more additional agents useful as pharmacological enhancers.

87. The method of claim 86, wherein said one or more additional agents as pharmacological enhancers is selected from the group consisting of ritonavir and cobicistat.

88. (canceled)
